(19) United States
(12) Patent Application Publication
(10) Pub. No.: US 2022/0160797 A1
(43) Pub. Date: May 26, 2022

(54) PROBIOTIC BACTERIA CAPABLE OF ADAPTIVE RESPONSE TO POMEGRANATE EXTRACT AND METHODS OF PRODUCTION AND USE THEREOF

(71) Applicant: North Carolina State University, Raleigh, NC (US)

(72) Inventors: Rodolphe Barrangou, Raleigh, NC (US); Sarah O'Flaherty, Raleigh, NC (US)

(21) Appl. No.: 17/442,776

(22) PCT Filed: Mar. 27, 2020
(86) PCT No.: PCT/US2020/025243

§ 371 Date: Sep. 24, 2021

Related U.S. Application Data
(60) Provisional application No. 62/827,480, filed on Apr. 1, 2019.

(51) Int. Cl. A61K 35/747; A23L 33/105; A23L 33/135; A61K 31/37; A61K 31/7048; A61K 36/185; C07K 14/335; C12N 1/20; C12N 15/01; C12N 9/24; C12P 17/06

(52) U.S. Cl. CPC A61K 35/747; A23L 33/105; A23L 33/135; A61K 31/37; A61K 31/7048; A23K 10/18; C07K 14/335; C12N 1/20; C12N 15/01; C12N 9/2402; C12P 17/06; A61K 36/185

(57) ABSTRACT

The invention relates to probiotic compositions, sachets, and nutritional supplements comprising a probiotic bacterium capable of an adaptive response resulting from growth in the presence of pomegranate extract, as well as methods of selecting, producing, and using thereof.

### FIELD OF THE INVENTION

The invention relates to probiotic compositions, sachets, and nutritional supplements comprising a probiotic bacterium capable of an adaptive response resulting from growth in the presence of pomegranate extract, as well as methods of selecting, producing, and using thereof.

### STATEMENT REGARDING ELECTRONIC FILING OF A SEQUENCE LISTING

A Sequence Listing in ASCII text format, submitted under 37 C.F.R. § 1.821, entitled 5051-944WO_ST25.txt, 12,032 bytes in size, generated on Mar. 24, 2020 and filed via EFS-Web, is provided in lieu of a paper copy. This Sequence Listing is hereby incorporated herein by reference into the specification for its disclosures.

### BACKGROUND OF THE INVENTION

A significant portion of the thousands of diet-derived known xenobiotic phytochemicals exhibit positive health effects in humans via anti-inflammatory, antiestrogenic, cardioprotective, anticarcinogenic, chemopreventative, neuroprotective, antimicrobial or antioxidants properties. However, it is often the case that phytochemicals occur as glyco-conjugates, and thus exhibit lower bioactivity and bioavailability than their aglycone derivatives, which are smaller in size and typically less polar. The deglycosylation of PGs may be a factor in modulating their biological activity. Recently, the health-impact of human gut microbiota (HGM)-mediated biotransformation of drug and diet-derived xenobiotics, including phytochemicals, has gained considerable interest, but knowledge of the metabolomechanisms and the therapeutic potential of HGM is limited.

Biotransformation of plant glycosides is a positive attribute of certain species of lactobacilli that impacts human health. Use of targeted strains of bacteria such as lactobacilli to positively influence the gut microbiota and help treat human disease are needed. These strains can have a direct effect on the gut microbiota or work though the biotransformation of drug and diet-derived xenobiotics, including phytochemicals. In addition to providing the benefit of biotransformation, technological advances that result in decreased fermentation times and higher biomasses are advantageous traits. Strain enhancement allows for improved lactobacilli strains and/or growth procedures that will impact nutrigenomics, therapeutic and human health applications.

### SUMMARY OF THE INVENTION

Provided herein are probiotic compositions comprising a probiotic bacterium capable of an adaptive response to pomegranate extract, and methods of selecting, producing, delivering, and using the same.

One aspect of the invention comprises a probiotic composition comprising a probiotic bacterium capable of an adaptive response, wherein the probiotic bacterium has been pre-cultured in a culture medium comprising pomegranate extract; and pomegranate extract.

A second aspect provides a method of selecting a bacterium capable of an adaptive response, comprising (a) pre-culturing a bacterium in a culture medium comprising pomegranate extract to produce a pre-cultured bacterial population; (b) culturing at least a portion of the pre-cultured bacterial population in a culture medium comprising pomegranate extract and determining if the pre-cultured bacterial population has a shorter time to lag phase and/or increased final optical density (OD) and/or increased tolerance to pomegranate extract; and (c) selecting the bacterium used to produce the bacterial population that has a shorter time to lag phase, and/or an increased final OD, and/or an increased tolerance to pomegranate extract, thereby selecting the bacterium capable of an adaptive response. In some embodiments, the invention provides a probiotic bacterium or bacterial strain capable of an adaptive response selected by the methods provided herein.

Another aspect of the invention provides method of adapting a bacterium capable of an adaptive response, comprising (a) pre-culturing a bacterium in a culture medium comprising pomegranate extract to produce a pre-cultured bacterial population; and (b) culturing at least a portion of the pre-cultured bacterial population in a culture medium comprising pomegranate extract, wherein the pre-cultured bacterial population exhibits a shorter time to lag phase and/or increased final optical density (OD) and/or increased tolerance to pomegranate extract. In some embodiments, the invention provides a pomegranate-adapted bacterium adapted by the methods provided herein.

Another aspect of the invention provides a method of increasing tolerance of a bacterial population to pomegranate extract in a culture medium, comprising culturing at least a portion of a bacterial population capable of an adaptive response in a culture medium comprising pomegranate extract to produce a pomegranate-adapted bacterial population, wherein the bacterial population capable of an adaptive response has been pre-cultured in a culture medium comprising pomegranate, thereby increasing the tolerance of the bacterial population to pomegranate extract in the culture medium.

A further aspect of the invention provides a method of producing a bacterial population having increased tolerance to pomegranate extract in a culture medium, comprising culturing at least a portion of a bacterial population capable of an adaptive response in a culture medium comprising pomegranate extract, wherein the bacterial population capable of an adaptive response has been pre-cultured in a culture medium comprising pomegranate, thereby producing a pomegranate-adapted bacterial population having increased tolerance to pomegranate extract in the culture medium.

Another aspect of the invention provides a method of decreasing time to lag phase of a bacterial population when cultured in a medium with or without pomegranate extract, comprising culturing at least a portion of a bacterial population capable of an adaptive response in a culture medium comprising pomegranate extract to produce a pomegranate-adapted bacterial population, wherein the bacterial population capable of an adaptive response has been pre-cultured in a culture medium comprising pomegranate, thereby decreasing the time to lag phase of the bacterial population when cultured in the medium with or without pomegranate extract.

An additional aspect of the invention provides a method of producing a bacterial population having decreased time to lag phase when grown in a culture medium with or without pomegranate extract, comprising culturing at least a portion of a bacterial population capable of an adaptive response in a culture medium comprising pomegranate extract, wherein the bacterial population capable of an adaptive response has been pre-cultured in a culture medium comprising pomegranate, thereby producing a pomegranate-adapted bacterial population having decreased time to lag phase when grown in the culture medium with or without pomegranate extract.

A further aspect of the invention provides a method of increasing final optical density (OD) of a bacterial population when cultured in a medium with or without pomegranate extract, comprising culturing at least a portion of a bacterial population capable of an adaptive response in a culture medium comprising pomegranate extract to produce a pomegranate-adapted bacterial population, wherein the bacterial population capable of an adaptive response has been pre-cultured in a culture medium comprising pomegranate, thereby increasing the final OD of the bacterial population when cultured in the medium with or without pomegranate extract.

An additional aspect of the invention provides a method of producing a bacterial population having an increased final optical density (OD) when grown in a culture medium with or without pomegranate extract, comprising culturing at least a portion of a bacterial population capable of an adaptive response in a culture medium comprising pomegranate extract, wherein the bacterial population capable of an adaptive response has been pre-cultured in a culture medium comprising pomegranate, thereby producing a bacterial population having increased final OD when grown in the culture medium with or without pomegranate extract.

Another aspect of the invention provides a method of producing a bacterial population with a reduced fermentation time to achieve a final optical density (OD), comprising culturing at least a portion of a bacterial population capable of an adaptive response in a culture medium comprising pomegranate extract to produce a bacterial population having reduced fermentation time to achieve a final OD compared to a control, wherein the bacterial population capable of an adaptive response has been pre-cultured in a culture medium comprising pomegranate.

An additional aspect of the invention provides a method of producing a probiotic bacterial population for the manufacture of a probiotic product, comprising culturing at least a portion of a pre-cultured probiotic bacterial population capable of an adaptive response in the presence of pomegranate extract (e.g., when grown in pomegranate extract) to produce a probiotic pomegranate-adapted bacterial population, wherein the probiotic bacterial population capable of an adaptive response has been pre-cultured in a culture medium comprising pomegranate.

Further provided herein are methods of delivering a probiotic composition to a subject, comprising administering to the subject the probiotic compositions and/or nutritional supplements of the invention.

An additional aspect of the invention provides a method of treating and/or reducing the risk of developing a disease in a subject in need thereof, comprising administering to the subject a therapeutically effective amount of a probiotic composition of the invention, and/or the nutritional supplements of the invention, thereby treating and/or reducing the risk of developing the disease in the subject. In some aspects, the disease may be a digestive disease, an inflammatory disease, cancer, or any combination thereof.

These and other aspects of the invention are set forth in more detail in the description of the invention below.

### BRIEF DESCRIPTION OF THE DRAWINGS

FIG. 1 provides a demonstration of the adaptive response for L. acidophilus NCFM and loss of the adaptive response after removal of pomegranate extract from the transfer media for the third and fourth transfer of four total transfers. Top graph; growth curves were performed in semi-defined medium (SDM) with 0.5% glucose. Bottom graph; growth curves were performed in SDM with 0.5% glucose plus pomegranate extract 400 μg/ml. Legend: (G.G): four transfers in SDM with 1% glucose; (G.P): two transfers in SDM with 1% glucose followed by two transfers in SDM with 1% glucose plus pomegranate extract 400 μg/ml; (P.G): two transfers in SDM with 1% glucose plus pomegranate extract 400 μg/ml followed by two transfers in SDM with 1% glucose; (P.P): four transfers in SDM with 1% glucose plus pomegranate extract 400 μg/ml.

FIGS. 2A-2C provide comparison growth curves of bacterial strains capable and incapable of an adaptive response when grown in the presence of pomegranate extract. FIG. 2A shows L. acidophilus NCFM (Lac NCFM), capable of adaptive response; FIG. 2B shows L. crispatus DNH-429 (Lcp DNH-429), incapable of adaptive response; FIG. 2C shows L. crispatus NCK1351 (Lcp NCK1351), incapable of adaptive response. Growth curves shown from culture after transfer into SDM with 1% glucose (left) or SDM with 1% glucose and 400 μg/ml pomegranate extract (right) following growth in a 0.5% glucose culture without pomegranate extract (“No POM”) or with 400 μg/ml (“POM 400”) or 800 μg/ml (“POM 800”) pomegranate extract.

FIGS. 3A-3C provide volcano plots for differential expression for each strain after growth in SDM with 0.5% glucose plus pomegranate extract compared to control SDM with 0.5% glucose. FIG. 3A shows Lac NCFM and Lga ATCC33323. FIG. 3B shows Lga NCK1340 and Lga NCK1342. FIG. 3C shows Lcr NCK1351 and Lcr DNH-429. Lac: Lactobacillus acidophilus; Lga: Lactobacillus gasseri; Lcr: Lactobacillus crispatus. Vertical lines to the left and right of center indicate the demarcation for genes that demonstrated a log₂ ratio >one and p valve <0.05.

FIG. 4 provides optical density (OD) measurements of lactobacilli strains and their growth phenotype when transferred into 1% glucose culture without pomegranate extract (“transfer in 1% G”) or into culture with pomegranate extract (“transfer in 1% G and POM 400”) after growth in a 0.5% glucose culture without pomegranate extract (“No POM”) or growth with 400 μg/ml (“POM 400”) or 800 μg/ml (“POM 800”) pomegranate extract.

FIG. 5 shows comparative growth curves of bacteria growing in three different 0.5% glucose media conditions (“SDM”, “POM 400”, “POM 800”), showing growth of L. acidophilus NCFM with no exposure to pomegranate extract (“POM”) (A), during first exposure to POM (B), or during second exposure to POM (C). Growth curves performed after initial expansion from frozen stocks in culture conditions, wherein (A) was assayed after grown in SDM from stock and never exposed to POM prior to assay, wherein (B) was grown in SDM from stock and then pre-cultured one round in SDM but never exposed to POM prior to assay, and wherein (C) was grown in SDM from stock and then pre-cultured one round in SDM with 400 μg/ml POM (i.e., first exposure to POM prior to assay). Top panel shows growth in SDM culture media alone. Middle panel shows growth in SDM supplemented with 400 μg/ml POM (“POM 400”). Bottom panel shows growth in SDM supplemented with 800 μg/ml POM (“POM 800”). Condition (A) demonstrates unadapted (e.g., SDM only) response. Condition (B) demonstrates first exposure (e.g., adapting, e.g., not-yet adapted) response in the presence of POM (e.g., when grown in pomegranate extract). Condition (C) demonstrates adapted (e.g., “primed’) response in the presence of POM.

FIGS. 6A-6B show concentrations of punicalagin (FIG. 6A) and ellagic acid (FIG. 6B) in proteomics assays of cell free supernatants and pellets from cultures from specific bacteria listed on x-axis.

FIG. 7 shows matrices of percent identity between bacterial strains NCK1342, NCK1340, NCK334, and NCK56 for three genes. Top matrix: gene dpT. Middle matrix: gene emrB. Bottom matrix: gene hsrA.

FIG. 8 provides a growth curve (OD600 nm) (upper panel) and graph (CFU/ml) (lower panel) of L. acidophilus NCFM in response to different media. First letter means the condition of the transfers and the second letter means the condition of the experiment (G: SDMGlu; P: SDMGlu+POM). Legend: (GG): two transfers in SDM with 1% glucose followed by growth in SDM with 0.5% glucose; (GP800): two transfers in SDM with 1% glucose followed by growth in SDM with 0.5% glucose plus pomegranate extract 800 μg/ml; (PG): two transfers in SDM with 1% glucose plus pomegranate extract 400 μg/ml followed by growth in SDM with 0.5% glucose; (PP800): two transfers in SDM with 1% glucose plus pomegranate extract 400 μg/ml followed by growth in SDM with 0.5% glucose plus pomegranate extract 800 μg/ml.

FIG. 9 provides a growth curve (OD600 nm) (upper panel) and graph (CFU/ml) (lower panel) of L. plantarum Lp-115 in response to different media. First letter means the condition of the transfers and the second letter means the condition of the experiment (G: SDMGlu; P: SDMGlu+POM). Legend: (GG): two transfers in SDM with 1% glucose followed by growth in SDM with 0.5% glucose; (GP800): two transfers in SDM with 1% glucose followed by growth in SDM with 0.5% glucose plus pomegranate extract 800 μg/ml; (PG): two transfers in SDM with 1% glucose plus pomegranate extract 400 μg/ml followed by growth in SDM with 0.5% glucose; (PP800): two transfers in SDM with 1% glucose plus pomegranate extract 400 μg/ml followed by growth in SDM with 0.5% glucose plus pomegranate extract 800 μg/ml.

FIG. 10 provides a growth curve (OD600 nm) (upper panel) and graph (CFU/ml) (lower panel) of L. rhamnosus GG in response to different media. First letter means the condition of the transfers and the second letter means the condition of the experiment (G: SDMGlu; P: SDMGlu+POM). Legend: (GG): two transfers in SDM with 1% glucose followed by growth in SDM with 0.5% glucose; (GP800): two transfers in SDM with 1% glucose followed by growth in SDM with 0.5% glucose plus pomegranate extract 800 μg/ml; (PG): two transfers in SDM with 1% glucose plus pomegranate extract 400 μg/ml followed by growth in SDM with 0.5% glucose; (PP800): two transfers in SDM with 1% glucose plus pomegranate extract 400 μg/ml followed by growth in SDM with 0.5% glucose plus pomegranate extract 800 μg/ml.

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

“Optional” or “optionally” means that the subsequently described event or circumstance can or cannot occur, and that the description includes instances where the event or circumstance occurs and instances where it does not.

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measureable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “increased,” “enhance,” “enhanced,” “enhancing,” and “enhancement” (and grammatical variations thereof) describe an elevation of at least about 5%, 10%, 15%, 20%, 25%, 50%, 75%, 100%, 150%, 200%, 300%, 400%, 500% or more as compared to a control.

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 35%, 50%, 75%, 80%, 85%, 90%, 95%, 97%, 98%, 99%, or 100% as compared to a control. In particular embodiments, the reduction can result in no or essentially no (i.e., an insignificant amount, e.g., less than about 10% or even 5%) detectable activity or amount.

“Bioactivity” or “bioactive” as used herein relates to the effects a given substance exerts on a living system, cell, or organism. Generally, bioactivity of a substance involves the uptake of the substance into a living system, cell, or organism, such that the substance can exert a physiological effect on that living system, cell, or organism. In some cases, a cell or organism can interact with a substance to increase the bioactivity of that substance in another cell or organism (e.g., symbiosis). Increases in bioactivity often correlate with increases in bioavailability. Bioactivity may be measured in a cell free system (i.e., in vitro) or in vivo.

“Bioavailability” or “bioavailable” as used herein relates to the degree and/or rate at which a substance (e.g., phytochemical) is absorbed into a living system, cell or organism, or is made available at a site of physiological activity. The term “bioavailability” as used herein can indicate the fraction of an orally administered dose that reaches the systemic circulation as an intact substance, taking into account both absorption and local metabolic degradation. As would be recognized by one of skill in the art based on the present disclosure, there are many factors that influence bioavailability of a substance, including but not limited to, the degree to which a substance is or is not glycosylated. In some cases, bioavailability may be associated with cell permeability, such that increases in cell permeability may lead to increases in bioavailability. Generally, increases in bioavailability of a substance may lead to uptake and metabolic utilization of that substance by a cell or organism, and may also facilitate the bioactivity of the substance. In some cases, a cell or organism can interact with a substance to increase the bioavailability of that substance in another cell or organism (e.g., symbiosis).

As used herein, the term “bacterium” or “probiotic bacterium” can refer to a single bacterial cell or a group of bacterial cells, and can be understood as and used interchangeably with the terms “bacterial strain,” “bacterial species,” and/or “bacterial population” as relevant to the context.

As used herein, the term “probiotic bacterium” further refers to a bacterium (e.g., a bacterium, bacterial strain, bacterial species, and/or bacterial population) whose presence may have beneficial effects on the health of a host (e.g., a subject, e.g., a mammalian subject e.g., a human subject). Non-pathogenic, commensal bacteria of the intestinal microbiota (also referred to as a “microbiome”) may be probiotic bacteria. The term “probiotics” may also be used to refer to any composition that may contain live probiotic bacteria and/or bacterially derived compounds. Probiotics may be delivered in any format, for example but not limited to, as probiotic bacteria mixed into a beverage, a nutritional bar, a fermented and/or non-fermented dairy product such as cheese, milk, yogurt, or ice cream, or any combination thereof.

As used herein, the term “prebiotic” and/or “prebiotic plant compound” refers to a dietary compound that is indigestible by, for example, a mammalian host (e.g., a human). The prebiotic, e.g., prebiotic plant glycoside (PG), may be converted by bacteria in the host gut microbiota (e.g., HGM) into a different compound (e.g., a bioactive aglycone) which may be digestible by the host. The small intestine is the primary site for absorption of nutrients and xenobiotics, which lends extra gravity to the metabolic activities of the HGM prevalent in this part of the gastrointestinal tract, where probiotic bacteria such as Lactobacilli and Bifidobacteria constitute an important part of the microbial population. Lactobacilli and Bifidobacteria may convert prebiotics such as plant glycosides into bioactive compounds such as aglycones using specialized uptake and deglycosylation machinery. The deglycosylated moieties of PGs that typically possess increased bioactivities as compared to the parent compounds are externalized, rendering them bioavailable to the host and other microbiota taxa. Carbohydrates like glycosides are mainly taken up by phosphotransferase system (PTS) transporters in Lactobacilli.

As used herein, the term “adaptive response” and/or “adapted response” refers to an response of a bacterium, bacterial strain, bacterial species, and/or bacterial population (e.g., a probiotic bacterium, strain, species, and/or population) resulting from growth in the presence of pomegranate extract (e.g., when grown in a media comprising pomegranate extract—“in the presence of” as used in this context means that the pomegranate is in the media in which the bacteria is grown) that is measurably different in subsequent cultures and educated by (e.g., “primed” by) a first exposure to pomegranate extract (e.g., naïve and/or unprimed, e.g., not previously pre-cultured in the presence of pomegranate extract, e.g., not previously grown in a media comprising pomegranate extract). A bacterium, bacterial strain, bacterial species, and/or bacterial population that is capable of an adaptive response and which has been pre-cultured in media comprising pomegranate may be referred to as a “pomegranate adapted” bacterium, bacterial strain, bacterial species, and/or bacterial population. An adaptive response of a bacterium when it is cultured in media comprising pomegranate extract may include, but is not limited to, adaptive/phenotypic features such as a reduced time to lag phase and/or an increased final optical density (OD). An adaptive response of a bacterium capable of an adaptive response to growth in the presence of pomegranate may be observed following the first culturing (i.e., pre-culture) of the bacterium in media comprising pomegranate and again when the bacterium is further cultured in a medium with or without pomegranate (e.g., either when the bacteria growing in media comprising pomegranate are transferred to new media with or without pomegranate or when fresh media with or without pomegranate is added to the bacteria growing in media comprising pomegranate). The reduced time to lag phase and/or increased final OD is determined as compared to a control (e.g., the same bacterium, bacterial strain, bacterial species, and/or bacterial population cultured in a culture medium comprising pomegranate but that was not pre-cultured in the presence of pomegranate extract). An adaptive response may also comprise altered bacterial gene expression and/or altered pattern of gene expression, including, but not limited, to the induction and/or enhancement of transporter and/or deglycosylation genes, and/or other genes involved in the metabolomechanisms of carbohydrate uptake and phytochemical biotransformation. In some embodiments, an adaptive response may comprise an increased tolerance to the presence of pomegranate extract. In some embodiments, an increased tolerance to pomegranate may comprise, but is not limited to, adaptive/phenotypic features of a pomegranate-adapted bacterium, bacterial strain, bacterial species, and/or bacterial population such as a reduced time to lag phase, an increased final OD, and/or altered gene expression when the pomegranate-adapted bacterium, bacterial strain, bacterial species, and/or bacterial population is further cultured in a medium with pomegranate. Reduced time to lag phase, increased final OD and/or altered gene expression is determined as compared to the same bacterium, bacterial strain, bacterial species, and/or bacterial population when cultured in a medium with pomegranate extract but which was not pre-cultured (e.g., not adapted) in the presence of pomegranate extract. In some embodiments, an adaptive response may be maintained by growth in a medium comprising pomegranate extract. In some embodiments, an adaptive response may be maintained by growth in a medium comprising an amount of pomegranate extract in a range from about 1 μg/ml to about 1000 μg/ml or any range or value therein (e.g., about 10 μg/ml to about 1000 μg/ml, about 50 μg/ml to about 1000 μg/ml, about 100 μg/ml to about 1000 μg/ml, about 200 μg/ml to about 1000 μg/ml, about 300 μg/ml to about 1000 μg/ml, about 400 μg/ml to about 1000 μg/ml, about 500 μg/ml to about 1000 μg/ml, about 10 μg/ml to about 100 μg/ml, about 10 μg/ml to about 500 μg/ml, about 50 μg/ml to about 200 μg/ml, about 50 μg/ml to about 500 μg/ml, and any range or value therein (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 550, 600, 650, 700, 750, 800, 850, 900, 950, 1000 μg/ml or any range or value therein)). In some embodiments, an adaptive response of a bacterium, bacterial strain, bacterial species, and/or bacterial population may be lost after growth in a medium without pomegranate for more than 24 hours.

By “pharmaceutically acceptable” or “physiologically acceptable” it is meant a material that is not toxic or otherwise undesirable, i.e., the material may be administered to a subject without causing any undesirable biological effects.

By the term “treat,” “treating,” or “treatment of” (or grammatically equivalent terms) it is meant that the severity of a subject's condition is reduced or at least partially improved or ameliorated and/or that some alleviation, mitigation or decrease in at least one clinical symptom is achieved and/or there is a delay in the progression of the condition and/or prevention or delay of the onset of a disease or disorder.

As used herein, the term “prevent,” “prevents,” or “prevention” (and grammatical equivalents thereof) refers to a delay in the onset of a disease or disorder or the lessening of symptoms upon onset of the disease or disorder. The terms are not meant to imply complete abolition of disease and encompass any type of prophylactic treatment that reduces the incidence of the condition or delays the onset and/or progression of the condition.

A “treatment effective” amount as used herein is an amount that is sufficient to provide some improvement or benefit to the subject. Alternatively stated, a “treatment effective” amount is an amount that provides some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject and/or reduce the risk of developing a disease, disorder and/or clinical symptoms in a subject relative to what would occur in the absence of the methods of the invention. Those skilled in the art will appreciate that the level of prevention need not be complete, as long as some benefit is provided to the subject.

The term “administering” or “administration” of a probiotic composition to a subject includes any route of introducing or delivering to a subject a compound to perform its intended function. Administration can be carried out by any suitable route, including orally, intranasally, parenterally (intravenously, intramuscularly, intraperitoneally, intracisternally, intrathecally, intraventricularly, or subcutaneously), or topically. Administration includes self-administration and administration by another.

A “therapeutically effective” amount as used herein is an amount that provides some improvement or benefit to the subject. Alternatively stated, a “therapeutically effective” amount is an amount that will provide some alleviation, mitigation, or decrease in at least one clinical symptom in the subject (e.g., in the case of cancer, reduction in tumor burden, prevention of further tumor growth, prevention of metastasis, or increase in survival time; or in the case of an inflammatory disease, reduction in inflammation). Those skilled in the art will appreciate that the therapeutic effects need not be complete or curative, as long as some benefit is provided to the subject.

As used herein, the terms “pre-culture” or “pre-culturing” refer to a first culturing of a bacterium (e.g., bacterium, bacterial strain, bacterial species, and/or bacterial population) in media comprising pomegranate extract. A bacterium (e.g., bacterium, bacterial strain, bacterial species, and/or bacterial population) that has been pre-cultured may be referred to as a pre-cultured or pomegranate-adapted bacterium (e.g., pre-cultured/pomegranate-adapted bacterium, bacterial strain, bacterial species, and/or bacterial population).

A “subject” of the invention may include any animal in need thereof. In some embodiments, a subject may be, for example, a mammal, a reptile, a bird, an amphibian, or a fish. A mammalian subject may include, but is not limited to, a laboratory animal (e.g., a rat, mouse, guinea pig, rabbit, primate, etc.), a farm or commercial animal (e.g., cattle, pig, horse, goat, donkey, sheep, etc.), or a domestic animal (e.g., cat, dog, ferret, gerbil, hamster etc.). In some embodiments, a mammalian subject may be a primate, or a non-human primate (e.g., a chimpanzee, baboon, monkey, gorilla, etc.). In some embodiments, a mammalian subject may be a human. In some embodiments, a bird may include, but is not limited to, a chicken, a duck, a turkey, a goose, a quail, a pheasant, a parakeet, a parrot, a macaw, a cockatoo, or a canary.

A “subject in need” of the methods of the invention can be a subject known to have a bodily discomfort and/or illness to which a probiotic composition may provide beneficial health effects, or a subject having an increased risk of developing the same (e.g., a subject having, for example, a digestive disease, an inflammatory disease, cancer, or any combination thereof).

### Probiotic Compositions

The present invention is directed to the field of microbiota research and therapy. In particular, the present invention provides compositions of probiotic bacteria capable of an adaptive response to pomegranate extract (e.g., resulting from growth in the presence of pomegranate extract). Pomegranate fruit is a dietary source of antioxidants and other phytochemicals, including polyphenols such as the ellagic and/or gallic acid derivative group of tannins called ellagitannins.

Thus, one aspect of the invention relates to a probiotic composition comprising: a probiotic bacterium capable of an adaptive response, wherein the probiotic bacterium has been pre-cultured in a culture medium comprising pomegranate; and pomegranate extract. In some embodiments, the composition may further comprise a prebiotic plant glycoside, wherein the probiotic bacterium is capable of converting the prebiotic plant glycoside into a bioactive aglycone. The prebiotic plant glycoside may be any known or putative prebiotic plant glycoside. Similarly, the bioactive aglycone may any known or putative aglycone, such as the bioactive aglycone formed from the deglycosylation of any known or putative prebiotic plant glycoside. In some embodiments, the prebiotic plant glycoside may include, but is not limited to, a punicalagin or a polyphenol ellagic acid. In some embodiments, the bioactive aglycone may include, but is not limited to, a urolithin.

In some embodiments, a composition provided herein may further comprise one or more pharmaceutically acceptable carriers and/or excipients. For example, pharmaceutically acceptable carriers or excipients can include various substances that facilitate the formation, digestion, and/or metabolism of a composition that includes a probiotic bacterium (e.g., a probiotic bacterium, strain, species, and/or population), pomegranate extract, and optionally a prebiotic plant glycoside. Pharmaceutically acceptable excipients and carriers can include, but are not limited to, one or more of cellulose, microcrystalline cellulose, mannitol, glucose, sucrose, trehalose, xylose, skim milk, milk powder, polyvinylpyrrolidone, tragacanth, acacia, starch, alginic acid, gelatin, dibasic calcium phosphate, stearic acid, croscarmellose, silica, polyethylene glycol, hemicellulose, pectin, amylose, amylopectin, xylan, arabinogalactan, polyvinylpyrrolidone, and combinations thereof. Carriers can include, but are not limited to, lactose, gum acacia, gelatin, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition, auxiliary, stabilizing, thickening, and coloring agents may also be used. The compositions of the invention may also be combined with various nontoxic, pharmaceutically acceptable carriers for tablets, pellets, capsules, troches, lozenges, aqueous or oily suspensions, dispersible powders or granules, suppositories, solutions, emulsions, suspensions, hard or soft capsules, caplets or syrups or elixirs and any other form suitable for use.

In some embodiments, a probiotic bacterium (e.g., bacterium, strain, species, and/or population) of the present invention may be adapted to growth in a medium comprising pomegranate extract (e.g., is a pomegranate-adapted bacterium, e.g., exhibits an adaptive response; e.g., has a reduced time to lag phase and/or an increased final OD and/or altered gene expression and/or increased tolerance to pomegranate extract in the growth medium) as compared to the probiotic bacterium that has not been adapted to growth in media comprising pomegranate extract. An altered gene expression of bacteria adapted to growth on media comprising pomegranate can comprise, but is not limited to, induction and/or enhancement of transporter and/or deglycosylation genes, and/or other genes involved in the metabolomechanisms of carbohydrate uptake and phytochemical biotransformation. In some embodiments, a probiotic bacterium capable of an adaptive response may have increased expression of transporter genes and/or glucosidase genes, optionally dtpT, emrB, hsrA, LBA1744, LGAS_RS08205, slpH3, bglA and/or any homologues thereof in response to growth in the presence of pomegranate extract during a first and/or subsequent exposure(s) to pomegranate extract.

A probiotic bacterium (e.g., bacterium, strain, species, and/or population) of the present invention can be any bacterium capable of probiotic features, e.g., beneficial health effects to the host (e.g., a subject, e.g., an animal subject, a mammalian subject, e.g., a human subject) that is capable of an adaptive response. In some embodiments, a probiotic bacterium may be an intestinal bacterium (e.g., a human intestinal bacterial species). In some embodiments, a probiotic bacterium useful with the invention may be from a bacterial genus including, but not limited to, Lactobacillus or Bifidobacterium. In some embodiments, a probiotic bacterium useful with the invention may be from a bacterial species including, but not limited to, L. acidophilus, L. casei, L. paracasei, L. crispatus, L. gasseri, L. plantarum, L. rhamnosus, L. salivarius, L. fermentum, L. reuteri, L. johnsonii, L. vaginalis, L. jensenii, L. helveticus, L. intestinalis, B. longum, B. lactis, B. infantis, or any combination thereof. In some embodiments, a probiotic bacterium may be, for example, L. acidophilus NCFM, L. acidophilus La-14, L. casei Lc11, L. crispatus NCK 1351, L. crispatus DNH-429, L. gasseri ATCC 33323, L. gasseri NCK 1138, L. gasseri NCK 1340, L. gasseri NCK 1341, L. gasseri NCK 1342, L. gasseri NCK 1343, L. gasseri Lg-36, L. plantarum Lp-115, L. plantarum Lpc-37, L. rhamnosus GG, L. salivarius Ls-33, or any combination thereof.

The amount of probiotic bacteria in a composition of the present invention may be any amount that is effective for delivering a beneficial health effect to the host (e.g., a subject, e.g., an animal subject, e.g., a mammalian subject, e.g., a human subject). In some embodiments, the amount of probiotic bacteria in the composition may be about 10⁸ cfu/g to about 10¹³ cfu/g, e.g., about 10⁸, 5×10⁸, 10⁹, 5×10⁹, 10¹⁰, 5×10¹⁰, 10¹¹, 5×10¹¹, 10¹², 5×10¹², or 10¹³ cfu/g, or any value or range therein.

Similarly, the amount of pomegranate extract in the composition may be any amount that is effective for delivering a beneficial health effect to the host (e.g., a subject, e.g., an animal subject, e.g., a mammalian subject, e.g., a human subject). In some embodiments, the amount of pomegranate extract in a composition of the invention may be about 50 μg/ml to about 2000 μg/ml, e.g., about 50, 100, 200, 300, 400, 500, 600, 700, 800, 900, 1000, 1100, 1200, 1300, 1400, 1500, 1600, 1700, 1800, 1900, or 2000 μg/ml, or any value or range therein, for example, about 50 μg/ml to about 1000 μg/ml, about 50 μg/ml to about 1500 μg/ml, about 100 μg/ml to about 1000 μg/ml, about 100 μg/ml to about 1500 μg/ml, or about 100 μg/ml to about 2000 μg/ml.

The pomegranate extract of the present invention may be any composition and/or mixture of components extracted from pomegranate fruit. In some embodiments, the pomegranate extract may comprise a polyphenol content of about 55% to about 65% gallic acid equivalents, e.g., about 55, 56, 57, 58, 59, 60, 61, 62, 63, 64, or 65%, or any value or range therein. In some embodiments, the pomegranate extract may comprise a polyphenol content of, for example, about 60% to about 65%. In some embodiments, the pomegranate extract may comprise a polyphenol content of about 61%. In some embodiments, a pomegranate extract may comprise oligomers of gallic acid, ellagic acid, and glucose. In some embodiments, the oligomers of gallic acid, ellagic acid, and glucose in a pomegranate extract may be composed of about 2 to about 10 repeating units, in any combination, e.g., about 2, 3, 4, 5, 6, 7, 8, 9, or 10 repeating units, in any combination. In some embodiments, about 65% to about 85% of a pomegranate extract may be comprised of gallic acid, ellagic acid, and glucose oligomers (e.g., about 65, 66, 67, 68, 69, 70, 71, 72, 73, 74, 75, 76, 77, 78, 79, 80, 81, 82, 83, 84, or 85%, or any value or range therein). In some embodiments, about 65% to about 80%, about 70% to about 80%, or about 70% to about 85% of the pomegranate extract may be comprise of gallic acid, ellagic acid, and glucose oligomers. In some embodiments, about 77% of a pomegranate extract may be comprised of gallic acid, ellagic acid, and glucose oligomers. In some embodiments, the pomegranate extract may comprise ellagitannins, free ellagic acid and anthocyanins. In some embodiments, the content of ellagitannins in a pomegranate extract may be about 15% to about 25%, e.g., about 15, 16, 17, 18, 19, 20, 21, 22, 23, 24, or 25%, or any value or range therein. In some embodiments, the content of ellagitannins in a pomegranate extract of the invention may be, for example, about 15% to about 22%, or about 15% to about 20%. In some embodiments, the content of ellagitannins in a pomegranate extract of the invention may be about 19%. Non-limiting examples of an ellagitannin includes punicalagin and/or punicalin. In some embodiments, the content of free ellagic acid in a pomegranate extract of the invention may be about 1% to about 10%, e.g., about 1, 2, 3, 4, 5, 6, 7, 8, 9, or 10%, or any value or range therein. In some embodiments, the content of free ellagic acid in a pomegranate extract of the invention may be, for example, about 1% to about 8%, or about 2% to about 6%. In some embodiments, the content of free ellagic acid in a pomegranate extract of the invention may be about 4%. In some embodiments, the content of anthocyanins in a pomegranate extract of the invention may be about 0% to about 4%, e.g., about 0, 1, 2, 3, or 4%, or any value or range therein. In some embodiments, the content of anthocyanins in a pomegranate extract of the invention may be, for example, about 0% to about 3%, or about 0% to about 2%. In some embodiments, the content of anthocyanins in a pomegranate extract of the invention may be about 0%. In some embodiments, the pomegranate extract may be a powder and further comprise ash, sugars, organic acids, and nitrogen. In some embodiments, the pomegranate extract powder may have a moisture content of less than about 10% (e.g., less than about 1, 2, 3, 4, 5, 6, 8, 9, 10%). In some embodiments, the content of ash in a pomegranate extract powder may be about 1% to about 5%, e.g., about 1, 1.5, 2, 2.5, 3, 3.5, 4, 4.5, or 5%, or any value or range therein. In some embodiments, the content of ash in a pomegranate extract powder may be, for example, about 1% to about 2%, about 1% to about 3%, about 1% to about 4%, about 1% to about 3%, about 2% to about 3%, about 2.5% to about 3%, or about 2% to about 4%. In some embodiments, the content of ash in a pomegranate extract powder may be about 2.2%. In some embodiments, the content of sugars in a pomegranate extract powder may be about 1% to about 5%, e.g., about 1, 1.5, 2, 2.5, 3, 3.5, 4, 4.5, or 5%, or any value or range therein. In some embodiments, the content of sugars in a pomegranate extract powder may be, for example, about 1% to about 3%, about 1% to about 4%, about 1% to about 3%, about 2% to about 3%, about 2.5% to about 3%, about 2.5% to about 3.5%, or about 2% to about 4%. In some embodiments, the content of sugars in a pomegranate extract powder may be about 2.9%. In some embodiments, the content of organic acids in a pomegranate extract may be about 1% to about 3%, e.g., about 1, 1.5, 2, 2.5, or 3%, or any value or range therein. In some embodiments, the content of organic acids in a pomegranate extract powder may be about 1% to about 2%, or about 1.5% to about 2.5%. In some embodiments, the content of organic acids in a pomegranate extract powder useful with this invention may be about 1.9%. In some embodiments, the content of nitrogen in a pomegranate extract powder may be about 0.1% to about 2%, e.g., about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2%, or any value or range therein. In some embodiments, the content of nitrogen in a pomegranate extract powder may be, for example, about 0.25% to about 0.75%, about 0.25% to about 1%, about 0.5% to about 0.9%, about 0.5% to about 1.0%, or about 0.5% to about 2.0%. In some embodiments, the content of nitrogen in a pomegranate extract powder may be about 0.7%.

In some embodiments, the pomegranate extract may comprise a polyphenol content of about 77% gallic acid, ellagic acid, and/or glucose oligomers (e.g., in any combination of 2-10 repeating units), a content of about 19% ellagitannins (e.g., punicalagins and punicalins), a content of about 4% free ellagic acid, a content of about 0% anthocyanins, and when in powder form, the pomegranate extract may further comprise about 2.2% ash, about 1.9% sugars, about 1.9% organic acids, about 0.7% nitrogen, and/or about 3.3% moisture. In some embodiments, a pomegranate extract may be POM WONDERFUL® pomegranate antioxidant extract.

The probiotic composition of the present invention may be formulated into a dosage form for delivery of the composition to a subject in need thereof. In some embodiments, the probiotic composition may be a dosage form that includes, but is not limited to, a tablet, a pellet, a hard capsule, a soft capsule, an emulsion, a powder, a dispersible powder, a lozenge, a troche, a chew, a gel, an aqueous or oily suspension, a spray, a granule, a suppository, a solution, a syrup, and/or an elixir.

In some embodiments, the present invention provides a packaging comprising the probiotic composition of the present invention. In some embodiments, the packaging can include, but is not limited to, a sachet, a blister pack, a bottle, a syringe, a nasal delivery device, and the like.

In some embodiments, the present invention provides a nutritional supplement comprising the probiotic composition of the present invention. Non-limiting examples of a nutritional supplement include a beverage, a nutritional bar, a fermented dairy product, a non-fermented dairy product, e.g., a juice, milk, yogurt, cheese, ice cream, or any combination thereof.

### Methods

The present invention further provides methods of selecting and producing a probiotic composition of the present invention.

In some embodiments, the present invention provides a method of selecting a bacterium (e.g., bacterium, bacterial strain, species, and/or population) capable of an adaptive response, the method comprising: (a) pre-culturing a bacterium (e.g., bacterium, bacterial strain, species, and/or population) in a culture medium comprising pomegranate extract to produce a pre-cultured bacterial population; (b) culturing at least a portion of the pre-cultured bacterial population in a culture medium comprising pomegranate extract and determining if the pre-cultured bacterial population has a shorter time to lag phase and/or increased final optical density (OD) and/or increased tolerance to pomegranate extract as compared to a control (e.g., the same bacterium cultured in a culture medium comprising pomegranate but that was not pre-cultured in the presence of pomegranate extract and/or the same bacterium that was pre-cultured in the presence of pomegranate extract and then cultured in a culture medium without pomegranate extract); and (c) selecting the bacterium used to produce the bacterial population that exhibits a shorter time to lag phase, and/or an increased final OD, and/or an increased tolerance to pomegranate extract as compared to the control, thereby selecting the bacterium (e.g., bacterial strain) capable of an adaptive response.

In some embodiments, the present invention provides a probiotic composition comprising a probiotic bacterium (e.g., bacterium, bacterial strain, species, and/or population) capable of an adaptive response selected by the methods provided herein.

In some embodiments, the present invention provides a method of adapting a bacterium capable of an adaptive response, comprising: (a) pre-culturing a bacterium (e.g., bacterium, bacterial strain, species, and/or population) in a culture medium comprising pomegranate extract to produce a pre-cultured bacterial population; and (b) culturing at least a portion of the pre-cultured bacterial population in a culture medium comprising pomegranate extract, wherein the pre-cultured bacterial population exhibits a shorter time to lag phase and/or increased final optical density (OD) and/or increased tolerance to pomegranate extract as compared to a control (e.g., the same bacterium cultured in a culture medium comprising pomegranate but that was not been pre-cultured in the presence of pomegranate extract and/or the same bacterium that was pre-cultured in the presence of pomegranate extract and then cultured in a culture medium without pomegranate extract).

In some embodiments, the present invention provides a probiotic composition comprising a probiotic pomegranate-adapted bacterium (e.g., bacterium, bacterial strain, species, and/or population) adapted by the methods provided herein.

In some embodiments, bacterial strains not capable (e.g., incapable) of an adaptive response may include, but are not limited to, L. crispatus DNH-429 L. crispatus NCK1351, L. delbreckii subsp. bulgaricus Lb-87, L. fermentum SBS-1, L. gasseri NCK99, L. gasseri NCK 1344, L. gasseri NCK1345 and L. reuteri 1E1

In some embodiments, a bacterium capable of an adapted response is selected according to the methods of the invention and used in additional methods of the invention, for example, for providing a bacterial population having a decreased time to lag phase (e.g., a reduced fermentation time to achieve lag phase), an increased final OD, or an increased tolerance to pomegranate in media.

In some embodiments, “culturing at least a portion” comprises transferring at least a portion of a bacterial population to culture media comprising pomegranate extract or culture media without pomegranate extract. In some embodiments, “culturing at least a portion” may comprise adding culture media comprising pomegranate extract or culture media without pomegranate extract to the bacterial population. As used herein, “at least a portion” of a bacterial population refers to a minimum amount of bacterial cells effective for growth expansion in a culture, e.g., at least 1, 10, 100, 1000, 10⁴, 10⁵, 10⁶, 10⁷, 10⁸ bacterial cells, e.g., about 1 to about 10⁸ bacterial cells. In some embodiments, at least a portion of a bacterial population may be expressed as colony forming units (cfu), such as, but not limited to, at least 1, 10, 100, 1000, 10⁴, 10⁵, 10⁶, 10⁷, 10⁸ total cfu, cfu/weight (e.g., cfu/g) or cfu/volume (e.g., cfu/ml). In some embodiments, at least a portion of a bacterial population may be expressed as a volume of culture liquid transferred and/or added to another culture, such as, but not limited to, at least 1 μl, 1 ml, 1 liter, or more from a culture medium comprising, for example, bacteria capable of an adaptive response, pre-cultured bacteria, pomegranate-adapted bacteria, or any other relevant bacteria and/or bacterial population.

In some embodiments, the invention provides a method of increasing tolerance of a bacterial population to pomegranate extract in a culture medium, comprising culturing at least a portion of a bacterial population capable of an adaptive response in a culture medium comprising pomegranate extract to produce a pomegranate-adapted bacterial population, wherein the bacterial population capable of an adaptive response has been pre-cultured in a culture medium comprising pomegranate, thereby increasing the tolerance of the bacterial population to the pomegranate extract in the culture medium as compared to a control (e.g., the bacterium, bacterial strain, species, and/or population capable of an adaptive response but which has not been cultured in the culture medium comprising pomegranate extract, e.g., not adapted). “Increased tolerance to the presence of pomegranate extract” refers to an adaptive response of a pomegranate-adapted bacterium that may comprise, but is not limited to, adaptive/phenotypic features such as a reduced time to lag phase, an increased final OD, and/or altered gene expression of the adapted bacterium, bacterial strain, species, and/or bacterial population as compared to the same bacterium, bacterial strain, species, and/or bacterial population that is not adapted (e.g., not pre-cultured in a medium with pomegranate before being cultured in a medium comprising pomegranate extract).

In some embodiments, the invention provides a method of increasing tolerance of a bacterial population to pomegranate extract in a culture medium, comprising pre-culturing a bacterial population capable of an adaptive response in a culture medium comprising pomegranate to produce a pre-cultured bacterial population, and culturing at least a portion of the pre-cultured bacterial population capable of an adaptive response in a culture medium comprising pomegranate extract, thereby producing a bacterial population having increased tolerance to pomegranate extract in a culture medium as compared to a control (e.g., the bacterium capable of an adaptive response but which has not been pre-cultured in the culture medium comprising pomegranate extract (e.g., not adapted)).

In some embodiments, the present invention provides a method of producing a bacterial population having increased tolerance to pomegranate extract in a culture medium, comprising culturing at least a portion of a bacterial population capable of an adaptive response in a culture medium comprising pomegranate extract, wherein the bacterial population capable of an adaptive response has been pre-cultured in a culture medium comprising pomegranate, thereby producing a pomegranate-adapted bacterial population having increased tolerance to pomegranate extract in the culture medium as compared to a control (e.g., the bacterium capable of an adaptive response but which has not been pre-cultured in the culture medium comprising pomegranate extract (e.g., not adapted)).

In some embodiments, the present invention provides a method of producing a bacterial population having increased tolerance to pomegranate extract in a culture medium, comprising pre-culturing a bacterial population capable of an adaptive response in a culture comprising pomegranate to produce a pre-cultured bacterial population, and culturing at least a portion of the pre-cultured bacterial population capable of an adaptive response in a culture medium comprising pomegranate extract, thereby producing a bacterial population having increased tolerance to pomegranate extract in a culture medium as compared to a control (e.g., the bacterium capable of an adaptive response but which has not been pre-cultured in the culture medium comprising pomegranate extract (e.g., not adapted)).

In some embodiments, the tolerance to pomegranate of the pomegranate-adapted bacterial population may be increased over a control by about 10% to about 200% or any range or value therein (e.g., the amount of pomegranate that is tolerated may be about 10% to about 100% more than is tolerated by a control) (e.g., about 10% to about 50%, about 10% to about 75%, about 10% to about 90%, about 20% to about 50%, about 20% to about 175%, about 20% to about 90%, about 20% to about 100%, about 50% to about 160%, about 50% to about 75%, about 50% to about 90%, about 50% to about 200% more than a control, or any range or value therein; e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200% more than a control or any range or value therein). In some embodiments, the tolerance to pomegranate of the pomegranate-adapted bacterial population may be increased over a control by about 2-fold to about 10-fold or any range or value therein (e.g., the amount of pomegranate that is tolerated may be about 2-fold to about 10-fold more than is tolerated by a control) (e.g., about 2-fold to about 9-fold, about 5-fold to about 10-fold, about 4-fold to about 8-fold, more than a control, or any range or value therein; e.g., about 2-fold, 3-fold, 4-fold, 5-fold, 6-fold, 7-fold, 8-fold, 9-fold, 10-fold, or more than a control or any range or value therein). In some embodiments, the tolerance to pomegranate of the pomegranate-adapted bacterial population may be expressed as a reduced time to lag phase and/or an increased final OD and/or altered gene expression to an equal amount of pomegranate extract in the growth medium as compared to a control (e.g., the bacterium capable of an adaptive response but which has not been cultured in media comprising pomegranate extract (e.g., not adapted)). In some embodiments, the tolerance to pomegranate of the pomegranate-adapted bacterial population may be an increased ability to grow (e.g., exhibits an adaptive response; e.g., has a reduced time to lag phase and/or an increased final OD and/or altered gene expression and/or increased tolerance to pomegranate extract in the growth medium) in a higher concentration of pomegranate extract as compared to a control (e.g., the bacterium capable of an adaptive response but which has not been cultured in media comprising pomegranate extract (e.g., not adapted)).

In some embodiments, the present invention provides a method of decreasing time to lag phase of a bacterial population when cultured in a culture medium with or without pomegranate extract, comprising culturing at least a portion of a bacterial population capable of an adaptive response in a culture medium comprising pomegranate extract to produce a pomegranate-adapted bacterial population), wherein the bacterial population capable of an adaptive response has been pre-cultured in a culture medium comprising pomegranate, thereby decreasing the time to lag phase of the bacterial population when cultured in the medium with or without pomegranate extract as compared to a control (e.g., the bacterium capable of an adaptive response but which has not been cultured in media comprising pomegranate extract (e.g., not adapted)).

In some embodiments, the present invention provides a method of decreasing time to lag phase of a bacterial population when cultured in a culture medium with or without pomegranate extract, comprising pre-culturing a bacterial population capable of an adaptive response in a culture comprising pomegranate to produce a pre-cultured bacterial population, and culturing at least a portion of the pre-cultured bacterial population capable of an adaptive response in a culture medium comprising pomegranate extract, thereby producing a bacterial population having a decreased time to lag phase when cultured in the medium with or without pomegranate extract as compared to a control (e.g., the bacterium capable of an adaptive response but which has not been pre-cultured in the culture medium comprising pomegranate extract (e.g., not adapted)).

In some embodiments, the present invention provides a method of producing a bacterial population having a decreased time to lag phase when grown in a culture medium with or without pomegranate extract, comprising culturing at least a portion of a bacterial population capable of an adaptive response in a culture medium comprising pomegranate extract, wherein the bacterial population capable of an adaptive response has been pre-cultured in a culture medium comprising pomegranate, thereby producing a pomegranate-adapted bacterial population having decreased time to lag phase when grown in the culture medium with or without pomegranate extract as compared to control (e.g., the bacterium capable of an adaptive response but which has not been cultured in the culture medium comprising pomegranate extract (e.g., not adapted)).

In some embodiments, the present invention provides a method of producing a bacterial population having a decreased time to lag phase when grown in a culture medium with or without pomegranate extract, comprising pre-culturing a bacterial population capable of an adaptive response in a culture medium comprising pomegranate, and culturing the pre-cultured bacterial population capable of an adaptive response in a culture medium comprising pomegranate extract, thereby producing a bacterial population having a decreased time to lag phase when compared to a control (e.g., the bacterium capable of an adaptive response but which has not been cultured in the culture medium comprising pomegranate extract (e.g., not adapted)).

In some embodiments, the present invention provides a method of increasing final optical density (OD) of a bacterial population when cultured in a medium with or without pomegranate extract, comprising culturing at least a portion of a bacterial population capable of an adaptive response in a culture medium comprising pomegranate extract to produce a bacterial population (e.g., a pomegranate adapted bacterial population), wherein the bacterial population capable of an adaptive response has been pre-cultured in a culture medium comprising pomegranate, thereby increasing the final OD of the bacterial population when cultured in a medium with pomegranate extract as compared to a control (e.g., the bacterium capable of an adaptive response that is cultured over the same time period in the same medium but without pomegranate extract (e.g., not adapted)).

In some embodiments, the present invention provides a method of increasing final optical density (OD) of a bacterial population when cultured in a medium with or without pomegranate extract, comprising pre-culturing a bacterial population capable of an adaptive response in a culture medium comprising pomegranate extract to produce a pre-cultured bacterial population, and culturing the pre-cultured bacterial population capable of an adaptive response in a culture medium comprising pomegranate extract, thereby producing a bacterial population having an increased final OD when cultured in the medium with or without pomegranate extract as compared to a control (e.g., the bacterium capable of an adaptive response that is cultured over the same time period in the same medium but without pomegranate extract (e.g., not adapted)).

In some embodiments, the present invention provides a method of producing a bacterial population having an increased final OD when grown in a culture medium with or without pomegranate extract, comprising culturing at least a portion of a bacterial population capable of an adaptive response in a culture medium comprising pomegranate extract, wherein the bacterial population capable of an adaptive response has been pre-cultured in a culture medium comprising pomegranate, thereby producing a bacterial population having increased final OD when grown in the culture medium with or without pomegranate extract as compared to a control (e.g., the bacterium capable of an adaptive response that is cultured over the same time period in the same medium but without pomegranate extract (e.g., not adapted)).

In some embodiments, the present invention provides a method of producing a bacterial population having an increased final OD when grown in a culture medium with or without pomegranate extract, comprising pre-culturing a bacterial population capable of an adaptive response in a culture comprising pomegranate extract to produce a pre-cultured bacterial population, and culturing at least a portion of the pre-cultured bacterial population capable of an adaptive response in a culture medium comprising pomegranate extract, thereby producing a bacterial population having an increased final OD when grown in the culture medium with or without pomegranate extract as compared to a control (e.g., the bacterium capable of an adaptive response but which has not been pre-cultured in the culture medium comprising pomegranate extract (e.g., not adapted)).

In some embodiments, an increase in final OD that is observed with the methods of the invention may be an increase over a control of about 1% to about 500%, or any range or value therein (e.g., about 1% to about 175%, about 1% to about 450%, about 1% to about 200%, about 5% to about 300%, about 5% to about 80%, about 5% to about 250%, about 5% to about 150%, about 10% to about 190%, about 10% to about 400%, about 10% to about 250%, about 25% to about 100%, about 25% to about 150%, about 25% to about 200%, about 25% to about 250%, about 50% to about 100%, about 50% to about 150%, about 50% to about 200%, about 50% to about 250%, about 40% to about 4′75%, about 25% to about 500%, or any value or range therein; e.g., about 1, 2, 3, 4, 5, 6, 7, 8, 9, 10, 15, 20, 25, 30, 35, 40, 45, 50, 55, 60, 75, 80, 85, 90, 95, 100, 110, 120, 130, 140, 150, 160, 170, 180, 190, 200, 210, 220, 230, 240, 245, 246, 247, 248, 249, 250, 275, 300, 350, 400, 450, 475, 490, 495, 496, 497, 498, 499, 500% or more than a control, or any range or value therein). In some embodiments, an increase in final OD that is observed with the methods of the invention may be an increase over a control of about 2-fold to about 100-fold and/or about 2-log₂ to about 10-log₂ or any range or value therein (e.g., about 2-fold to about 100-fold and/or about 2-log₂ to about 10-log₂ more than a control) (e.g., about 2-fold to about 90-fold, about 5-fold to about 100-fold, about 4-fold to about 96-fold, about 2-log₂ to about 10-log₂, about 4-log₂ to about 8-log₂ more than a control, or any range or value therein; e.g., about 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 95-fold, 100-fold, or more, or, e.g., about 2-log₂, 3-log₂, 4-log₂, 5-log₂, 6-log₂, 7-log₂, 8-log₂, 9-log₂, 10-log₂, or more than a control or any range or value therein).

In some embodiments, the present invention provides a method of producing a bacterial population with a reduced fermentation time to achieve a final optical density (OD), comprising culturing at least a portion of a bacterial population capable of an adaptive response in a culture medium comprising pomegranate extract to produce a bacterial population having reduced fermentation time to achieve a final OD compared to a control, wherein the bacterial population capable of an adaptive response has been pre-cultured in a culture medium comprising pomegranate (e.g., wherein a control may be e.g., the bacterium capable of an adaptive response but which has not been pre-cultured in the culture medium comprising pomegranate extract (e.g., not adapted)).

In some embodiments, the present invention provides a method of producing a bacterial population with a reduced fermentation time to achieve a final optical density (OD), comprising pre-culturing a bacterial population capable of an adaptive response in a culture comprising pomegranate extract to produce a pre-cultured bacterial population, and culturing at least a portion of the pre-cultured bacterial population capable of an adaptive response in a culture medium comprising pomegranate extract, thereby producing a bacterial population having reduced fermentation time to achieve a final OD compared to a control (e.g., the bacterium capable of an adaptive response but which has not been pre-cultured in the culture medium comprising pomegranate extract (e.g., not adapted)). In some embodiments, the methods disclosed herein may further comprise culturing at least a portion of the bacterial population having reduced fermentation time in an additional culture medium with or without pomegranate extract. In some embodiments, the methods disclosed herein may further comprise collecting the bacterial population (e.g., the probiotic pomegranate-adapted bacterial population) having reduced fermentation time for the manufacture of a probiotic product.

In some embodiments, a final OD of a population may be achieved in at least about 4 hours less time) (e.g., about 4 hours to about 10 hours less time; e.g., about 4, 4.5, 5, 5.5, 6, 6.5, 7, 7.5, 8, 8.5, 9, 9.5 or 10 hours, and any value or range therein) (e.g., a reduced fermentation time than a control (e.g., the same bacterium that has not been grown in the presence of pomegranate (e.g., for the control, the bacterium was never cultured in the presence of pomegranate or at least the immediately prior culture medium did not comprise pomegranate).

In some embodiments, the invention provides a method of producing a probiotic bacterial population for the manufacture of a probiotic product, comprising culturing at least a portion of a pre-cultured probiotic bacterial population capable of an adaptive response in the presence of pomegranate extract to produce a probiotic pomegranate-adapted bacterial population, wherein the probiotic bacterial population capable of an adaptive response has been pre-cultured in a culture medium comprising pomegranate. In some embodiments, the invention provides a method of producing a probiotic bacterial population for the manufacture of a probiotic product, comprising pre-culturing a probiotic bacterial population capable of an adaptive response in a culture medium comprising pomegranate extract to produce a pre-cultured bacterial population capable of an adaptive response; and culturing at least a portion of the pre-cultured probiotic bacterial population capable of an adaptive response in a culture medium comprising pomegranate extract, thereby producing a probiotic pomegranate-adapted bacterial population. In some embodiments, the method may further comprise culturing at least a portion of the pomegranate adapted bacterial population in a culture medium with or without pomegranate extract to produce a probiotic bacterial population. In some embodiments, wherein culturing at least a portion of the pomegranate-adapted bacterial population comprises transferring to a medium without pomegranate extract, the transfer may be directly from a medium comprising pomegranate extract to a medium without pomegranate extract (e.g., with no intervening transfers to media without pomegranate extract). In some embodiments, wherein the pomegranate-adapted bacterial population was previously cultured in a medium without pomegranate extract, the method may further comprise culturing at least a portion of the pomegranate-adapted bacterial population in a medium with pomegranate extract (e.g., with no intervening transfers to media without pomegranate extract). In some embodiments, the method may further comprise collecting the probiotic bacterial population (e.g., the probiotic pomegranate-adapted bacterial population) for the manufacture of a probiotic product.

In some embodiments, a culture medium comprising pomegranate extract may further comprise a carbohydrate. The carbohydrate may be any carbohydrate that is an appropriate source for growth of the probiotic bacterium. In some embodiments, the carbohydrate may be a sugar and/or oligosaccharide. In some embodiments, the carbohydrate may be glucose, lactose, sucrose, fructose, galactose, trehalose, raffinose, or a fructooligosaccaride. The carbohydrate may be present in a medium comprising pomegranate extract in any amount sufficient for the growth of the probiotic bacterium, e.g., from at least about 0.25% to about 3%; e.g., about 0.25, 0.5, 0.75, 1, 1.25, 1.5, 1.75, 2, 2.25, 2.5, 2.75, or 3%, or any value or range therein. In some embodiments, the carbohydrate may be present in a medium comprising pomegranate extract from, for example, at least about 0.25% to about 2%, or at least about 0.25% to about 1%. In some embodiments, the carbohydrate present in a medium comprising pomegranate extract may be glucose in the amount of about 0.5%.

In some embodiments, a culture medium comprising pomegranate extract may comprise about 50 to about 2000 μg/ml pomegranate extract; e.g., about 50, 75, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 μg/ml or any value or range therein. In some embodiments, a culture medium comprising pomegranate extract may comprise, for example, about 50 μg/ml to about 500 μg/ml, about 50 μg/ml to about 1000 μg/ml, about 50 μg/ml to about 1500 μg/ml, about 100 μg/ml to about 500 μg/ml, about 100 μg/ml to about 1000 μg/ml, about 100 μg/ml to about 1500 μg/ml, about 100 μg/ml to about 2000 μg/ml, about 200 μg/ml to about 500 μg/ml, about 200 μg/ml to about 1000 μg/ml, about 200 μg/ml to about 1500 μg/ml, about 200 μg/ml to about 2000 μg/ml, about 300 μg/ml to about 500 μg/ml, about 300 μg/ml to about 1000 μg/ml, about 300 μg/ml to about 1500 μg/ml, or about 300 μg/ml to about 2000 μg/ml pomegranate extract. In some embodiments, a culture medium comprising pomegranate extract may comprise about 400 μg/ml pomegranate extract.

The pomegranate extract of the present invention may be any composition and/or mixture of components extracted from pomegranate fruit. In some embodiments, the pomegranate extract may comprise a polyphenol content of about 55% to about 65% gallic acid equivalents, e.g., about 55, 56, 57, 58, 59, 60, 61, 62, 63, 64, or 65%, or any value or range therein. In some embodiments, the pomegranate extract may comprise a polyphenol content of, for example, about 60% to about 65%. In some embodiments, the pomegranate extract may comprise a polyphenol content of about 61%. In some embodiments, a pomegranate extract may comprise oligomers of gallic acid, ellagic acid, and glucose. In some embodiments, the oligomers of gallic acid, ellagic acid, and glucose in a pomegranate extract may be composed of about 2 to about 10 repeating units, in any combination, e.g., about 2, 3, 4, 5, 6, 7, 8, 9, or 10 repeating units, in any combination. In some embodiments, about 65% to about 85% of a pomegranate extract may be comprised of gallic acid, ellagic acid, and glucose oligomers (e.g., about 65, 66, 67, 68, 69, 70, 71, 72, 73, 74, 75, 76, 77, 78, 79, 80, 81, 82, 83, 84, or 85%, or any value or range therein). In some embodiments, about 65% to about 80%, about 70% to about 80%, or about 70% to about 85% of the pomegranate extract may be comprise of gallic acid, ellagic acid, and glucose oligomers. In some embodiments, about 77% of a pomegranate extract may be comprised of gallic acid, ellagic acid, and glucose oligomers. In some embodiments, the pomegranate extract may comprise ellagitannins, free ellagic acid and anthocyanins. In some embodiments, the content of ellagitannins in a pomegranate extract may be about 15% to about 25%, e.g., about 15, 16, 17, 18, 19, 20, 21, 22, 23, 24, or 25%, or any value or range therein. In some embodiments, the content of ellagitannins in a pomegranate extract of the invention may be, for example, about 15% to about 22%, or about 15% to about 20%. In some embodiments, the content of ellagitannins in a pomegranate extract of the invention may be about 19%. Non-limiting examples of an ellagitannin includes punicalagin and/or punicalin. In some embodiments, the content of free ellagic acid in a pomegranate extract of the invention may be about 1% to about 10%, e.g., about 1, 2, 3, 4, 5, 6, 7, 8, 9, or 10%, or any value or range therein. In some embodiments, the content of free ellagic acid in a pomegranate extract of the invention may be, for example, about 1% to about 8%, or about 2% to about 6%. In some embodiments, the content of free ellagic acid in a pomegranate extract of the invention may be about 4%. In some embodiments, the content of anthocyanins in a pomegranate extract of the invention may be about 0% to about 4%, e.g., about 0, 1, 2, 3, or 4%, or any value or range therein. In some embodiments, the content of anthocyanins in a pomegranate extract of the invention may be, for example, about 0% to about 3%, or about 0% to about 2%. In some embodiments, the content of anthocyanins in a pomegranate extract of the invention may be about 0%. In some embodiments, the pomegranate extract may be a powder and further comprise ash, sugars, organic acids, and nitrogen. In some embodiments, the pomegranate extract powder may have a moisture content of less than about 10% (e.g., less than about 1, 2, 3, 4, 5, 6, 8, 9, 10%). In some embodiments, the content of ash in a pomegranate extract powder may be about 1% to about 5%, e.g., about 1, 1.5, 2, 2.5, 3, 3.5, 4, 4.5, or 5%, or any value or range therein. In some embodiments, the content of ash in a pomegranate extract powder may be, for example, about 1% to about 2%, about 1% to about 3%, about 1% to about 4%, about 1% to about 3%, about 2% to about 3%, about 2.5% to about 3%, or about 2% to about 4%. In some embodiments, the content of ash in a pomegranate extract powder may be about 2.2%. In some embodiments, the content of sugars in a pomegranate extract powder may be about 1% to about 5%, e.g., about 1, 1.5, 2, 2.5, 3, 3.5, 4, 4.5, or 5%, or any value or range therein. In some embodiments, the content of sugars in a pomegranate extract powder may be, for example, about 1% to about 3%, about 1% to about 4%, about 1% to about 3%, about 2% to about 3%, about 2.5% to about 3%, about 2.5% to about 3.5%, or about 2% to about 4%. In some embodiments, the content of sugars in a pomegranate extract powder may be about 2.9%. In some embodiments, the content of organic acids in a pomegranate extract may be about 1% to about 3%, e.g., about 1, 1.5, 2, 2.5, or 3%, or any value or range therein. In some embodiments, the content of organic acids in a pomegranate extract powder may be about 1% to about 2%, or about 1.5% to about 2.5%. In some embodiments, the content of organic acids in a pomegranate extract powder useful with this invention may be about 1.9%. In some embodiments, the content of nitrogen in a pomegranate extract powder may be about 0.1% to about 2%, e.g., about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2%, or any value or range therein. In some embodiments, the content of nitrogen in a pomegranate extract powder may be, for example, about 0.25% to about 0.75%, about 0.25% to about 1%, about 0.5% to about 0.9%, about 0.5% to about 1.0%, or about 0.5% to about 2.0%. In some embodiments, the content of nitrogen in a pomegranate extract powder may be about 0.7%.

In some embodiments, the pomegranate extract may comprise a polyphenol content of about 77% gallic acid, ellagic acid, and/or glucose oligomers (e.g., in any combination of 2-10 repeating units), a content of about 19% ellagitannins (e.g., punicalagins and punicalins), a content of about 4% free ellagic acid, a content of about 0% anthocyanins, and when in powder form, the pomegranate extract may further comprise about 2.2% ash, about 1.9% sugars, about 1.9% organic acids, about 0.7% nitrogen, and/or about 3.3% moisture. In some embodiments, a pomegranate extract may be POM WONDERFUL® pomegranate antioxidant extract.

In some embodiments, the invention provides a probiotic bacterium that is adapted to growth in a medium comprising pomegranate extract (e.g., is a pomegranate-adapted bacterium, e.g., exhibits an adaptive response; e.g., has a reduced time to lag phase and/or an increased final optical density and/or altered gene expression and/or increased tolerance to pomegranate extract in the growth medium; e.g., a pomegranate adapted bacterium) as compared to a control (e.g., the same probiotic bacterium that has not been adapted to growth in media comprising pomegranate extract). In some embodiments, altered gene expression of a pomegranate adapted bacterium may comprise, but is not limited to, induction and/or enhancement of transporter and/or deglycosylation genes, and/or other genes involved in the metabolomechanisms of carbohydrate uptake and phytochemical biotransformation. In some embodiments, the probiotic bacterium adapted for growth in media comprising pomegranate may have increased expression of transporter genes and/or glucosidase genes, optionally dtpT, emrB, hsrA, LBA1744, LGAS_RS08205, slpH3, bglA and/or any homologues thereof.

A probiotic bacterium (e.g., bacterium, strain, species, and/or population) useful with this invention may be any bacterium, e.g., any bacterial genus, bacterial species or bacterial strain, capable of probiotic features, e.g., beneficial health effects to the host (e.g., a subject, e.g., an animal subject, e.g., a mammalian subject, e.g., a human subject). In some embodiments, a probiotic bacterium may be an intestinal bacterium (e.g., a human intestinal bacterial species). In some embodiments, a probiotic bacterium useful with the invention may be from a bacterial genus including, but not limited to, Lactobacillus or Bifidobacterium. In some embodiments, a probiotic bacterium useful with the invention may be from a bacterial species including, but not limited to, L. acidophilus, L. casei, L. paracasei, L. crispatus, L. gasseri, L. plantarum, L. rhamnosus, L. salivarius, L. fermentum, L. reuteri, L. johnsonii, B. longum, B. lactis, B. infantis, or any combination thereof. Non-limiting examples of bacterial strains useful with this invention include L. acidophilus NCFM, L. acidophilus La-14, L. casei Lc11, L. crispatus NCK 1351, L. crispatus DNH-429, L. gasseri ATCC 33323, L. gasseri NCK 1138, L. gasseri NCK 1340, L. gasseri NCK 1341, L. gasseri NCK 1342, L. gasseri NCK 1343, L. gasseri Lg-36, L. plantarum Lp-115, L. plantarum Lpc-37, L. rhamnosus GG, L. salivarius Ls-33, or any combination thereof.

The present invention further provides methods of using a probiotic composition of the present invention.

In some embodiments, the present invention provides a method of delivering a probiotic composition to a subject in need thereof, comprising administering to the subject (e.g., a therapeutically effective amount of) a probiotic composition or a nutritional supplement comprising the probiotic composition of the present invention. In some embodiments, the probiotic composition of the invention may be packaged in single or multiple doses in sachets, blister packs, syringes, nasal delivery devices, or the like, for delivery to a subject.

In some embodiments, the invention provides a method of treating and/or reducing the risk of developing a disease in a subject in need thereof, comprising administering to the subject (e.g., a therapeutically effective amount of) a probiotic composition or a nutritional supplement comprising a probiotic composition of the present invention, thereby treating and/or reducing the risk of developing the disease in the subject. In some embodiments, the disease in a subject in need thereof may be a digestive disease, an inflammatory disease, cancer, or any combination thereof. In some embodiments, the disease may be colitis, Crohn's disease, ulcerative colitis, inflammatory bowel disease (IBD), irritable bowel syndrome (IBS), pouchitis, gastrointestinal cancer, or any combination thereof.

In some embodiments, a composition of the invention may be administered to the subject in a therapeutically effective amount, as that term is defined above. A therapeutically effective dosage of any specific composition will vary somewhat from composition to composition, and subject to subject, and will depend upon the condition of the subject and the route of delivery. As a general proposition, a dosage of about 10⁸ cfu/g to about 10¹³ cfu/g of probiotic bacteria per gram of the composition will have therapeutic efficacy.

The invention will now be described with reference to the following examples. It should be appreciated that these examples are not intended to limit the scope of the claims to the invention, but are rather intended to be exemplary of certain embodiments. Any variations in the exemplified methods that occur to the skilled artisan are intended to fall within the scope of the invention.

### EXAMPLES

Lactobacilli strains were screened for their ability to grow in the presence of pomegranate extract and to determine the biotransformation ability of lactobacilli strains. Results are provided relating to the growth of lactobacilli in the presence of pomegranate extract, adaptation of specific lactobacilli strains for growth enhancement after transfers in medium containing pomegranate extract, and RNA-seq analysis of select lactobacilli after growth in media containing pomegranate extract compared to growth in media without pomegranate extract.

### Example 1: Growth of Lactobacilli in the Presence of Pomegranate Extract

Initial growth assays showed that pomegranate extract did not support growth of lactobacilli strains in the absence of an additional sugar source. Semi-defined medium (SDM; Kimmel and Roberts, 1998. Intl. J. Food. Microbiol. 40:87-92) with 0.5% (w/v) glucose used for growth assays. Typically growth medium contains 2% glucose but to avoid masking the effect of the pomegranate extract, less glucose was used here.

| SDM Ingredients | g/L |
|---|---|
| Glucose | |
| Tween 80 | 1 |
| Ammonium citrate | 2 |
| Sodium acetate | 5 |
| MgSO4 ·7H20 | 0.1 |
| MnSO4 | 0.05 |
| K2HPO4 | 2 |
| Yeast Nitrogen Base | 5 |
| Casitone | 10 |

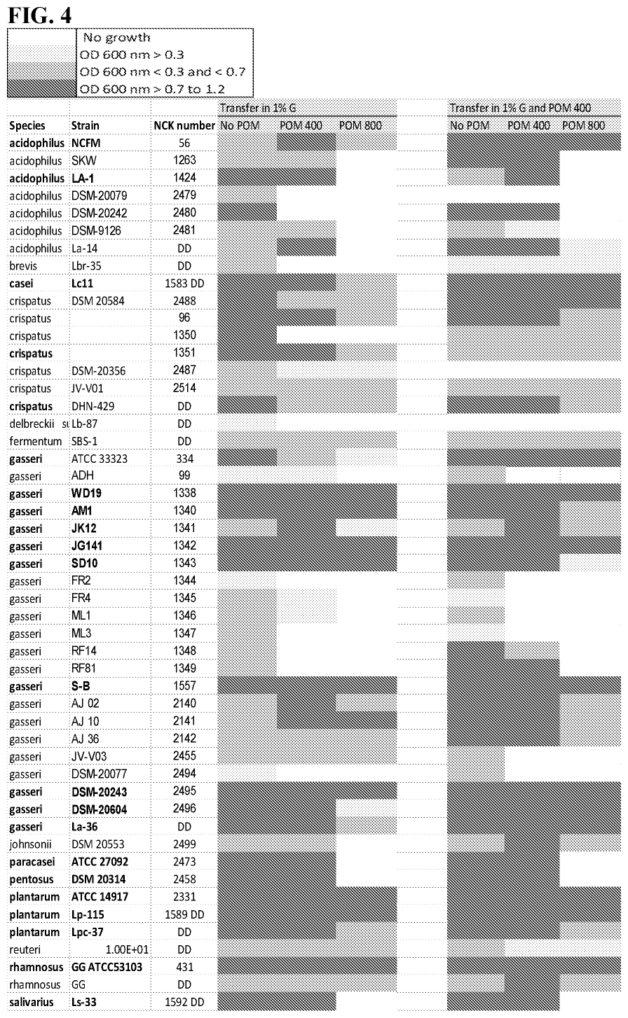

Preliminary growth assays with a subset set of lactobacilli showed varied results depending both on the species and on the strain used. Transfers of the cultures in the presence of pomegranate extract (400 μg/ml) prior to growth assays were then performed to determine if the transfers would impact growth assays over a 24 hour period. In total, 50 strains of lactobacilli were tested (Table 1) and again depending on the strain or species of lactobacilli the transfers in pomegranate extract prior to growth assays had a marked effect not just on growth in medium containing pomegranate extract but also in some cases in control media (FIG. 4). This response was termed the “adaptive response.” In particular Lactobacillus acidophilus NCFM (NCK56), and Lactobacillus gasseri ATCC 33323 (NCK334) demonstrated an adaptive response (FIG. 4). Based on the results of the growth assays, strains were selected for RNA-seq analysis and targeted proteomics). In addition, strains were selected on the basis of commercial potential.

### Example 2: Adaptation of Specific Lactobacilli Strains for Growth Enhancement after Transfers in Media Containing Pomegranate Extract

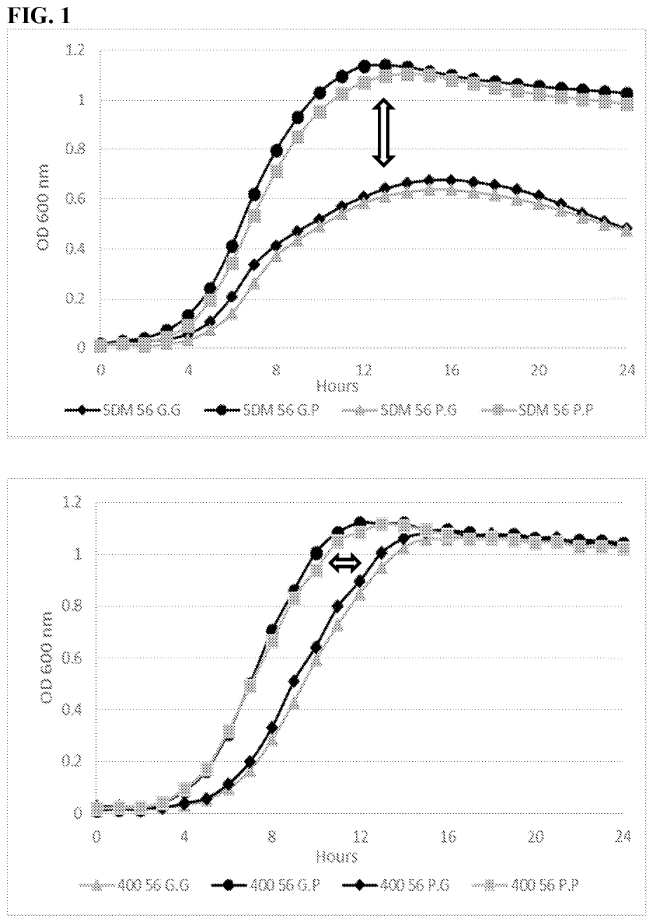

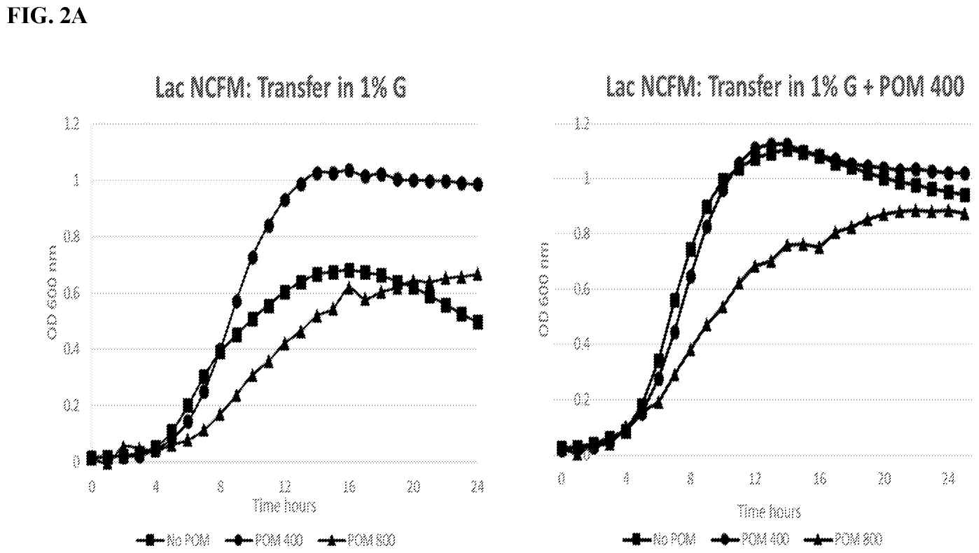

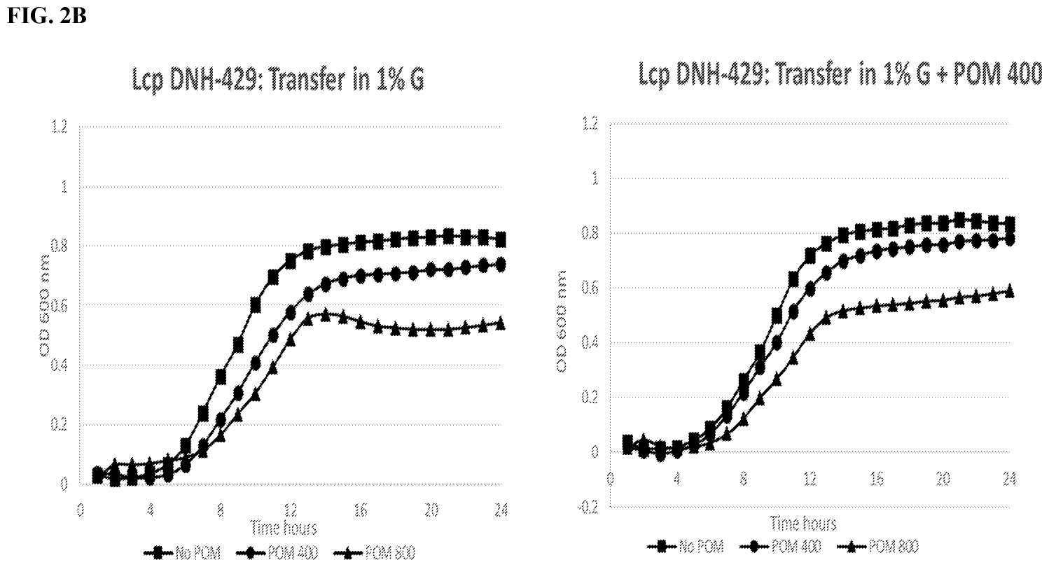

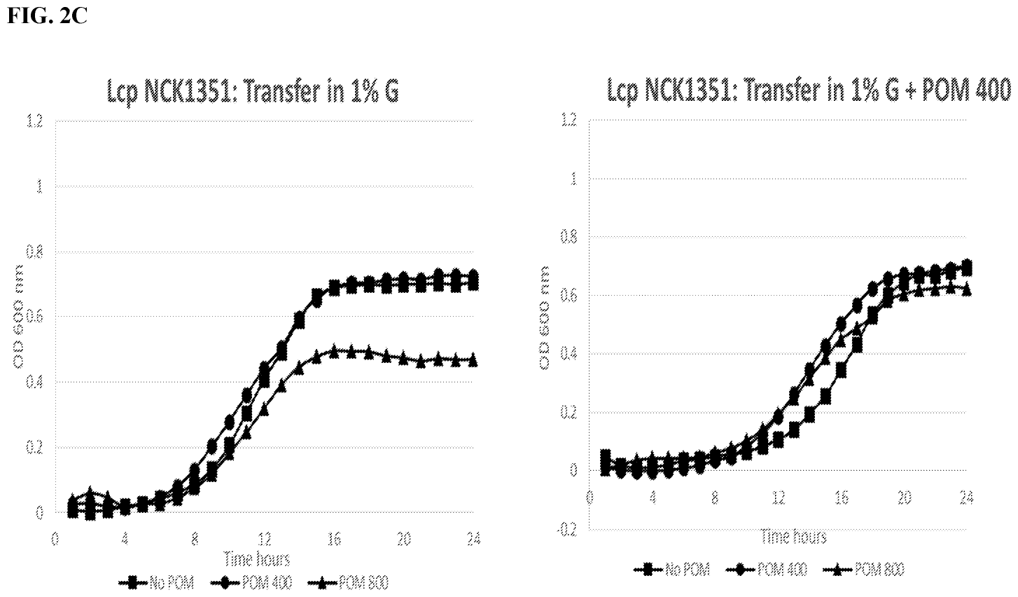

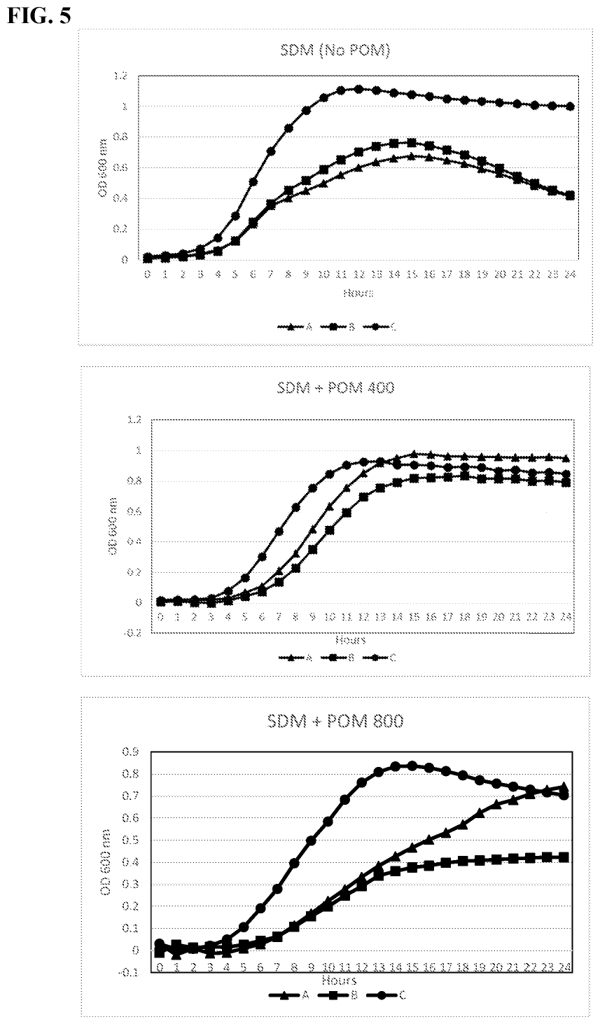

In the case of L. acidophilus NCFM, cells that had undergone the adaptive response showed better growth in control media and shortened lag phase times in the presence of pomegranate extract (FIG. 1). To determine if the adaptive response was lost after transfer to media without pomegranate extract after initial transfers to media with pomegranate extract, L. acidophilus NCFM was transferred four times in SDM with no pomegranate extract (control), four times in SDM with pomegranate extract (expected adaptive phenotype observed), transferred twice in SDM with no pomegranate extract and then twice in SDM with pomegranate extract (adaptive phenotype observed) and finally twice in SDM with pomegranate extract and then twice in SDM with no pomegranate extract (adaptive phenotype lost). This data shows that when pomegranate extract was removed from the media the adaptive response was lost (FIG. 1). In fact, in the case of L. acidophilus NCFM, just one transfer to media without pomegranate extract after two transfers to media with pomegranate extract (adaptive response) was sufficient for the bacterial population to lose the adaptive response. In comparison, FIGS. 2A-2C provide growth curves of bacteria that did and did not demonstrate an adaptive response to pomegranate extract. FIG. 5 provides direct comparison growth curves of an exemplar adaptable microbe (L. acidophilus NCFM) in conditions of no prior exposure to pomegranate extract (A), growth during first exposure to pomegranate extract (e.g., adapting, e.g., not yet adapted) (B), and growth during second exposure to pomegranate extract following first exposure at 400 m/ml pomegranate extract (e.g., post adaptation, e.g., adapted growth) (C). Growth curves performed in final media conditions of SDM alone (top panel) SDM with 400 m/ml (middle panel), or SDM with 800 m/ml (bottom panel). This constitutes novel, unexpected and valuable insights for the formulation of probiotic products that may be used in combination with pomegranate extract.

### Example 3: RNA-Seq Analysis of Select Lactobacilli after Growth in Media Containing Pomegranate Extract Compared to Media without Pomegranate Extract

Select lactobacilli strains, L. acidophilus NCFM, L. gasseri ATCC33323, NCK1340 and NCK1342 and L. crispatus NCK1351 and DNH-429 were transferred twice in SDM with 1% glucose and pomegranate extract (400 μg/ml) and then transferred and grown to mid-log phase in SDM with 0.5% glucose or SDM with 0.5% glucose and pomegranate extract (400 μg/ml). Total RNA was isolated from biological replicates of each culture and RNA sequenced. For each strain, differential gene expression analysis was performed to compare gene expression between cells grown in the control (SDM, 0.5% glucose) and test medium (SDM, 0.5% glucose with pomegranate extract).

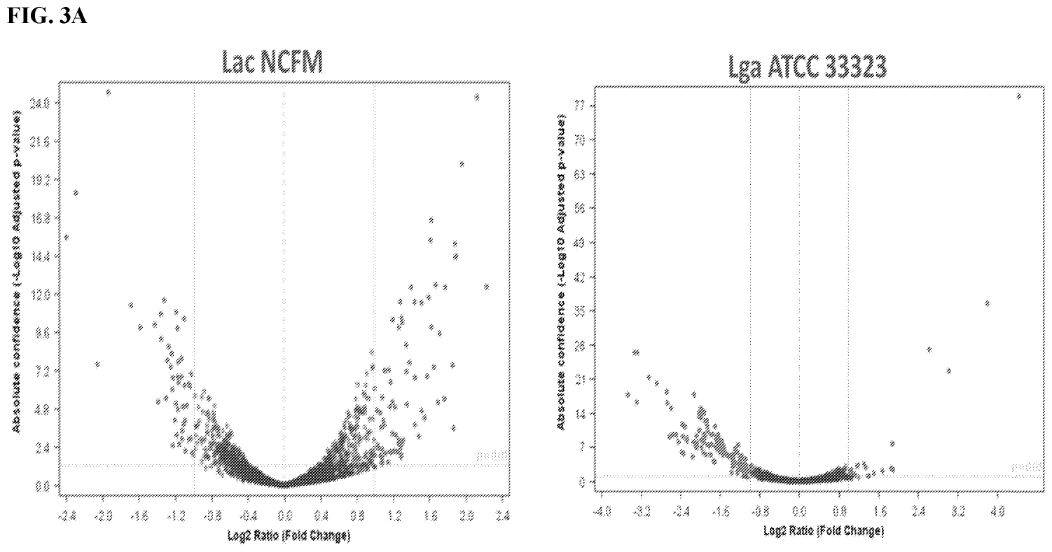

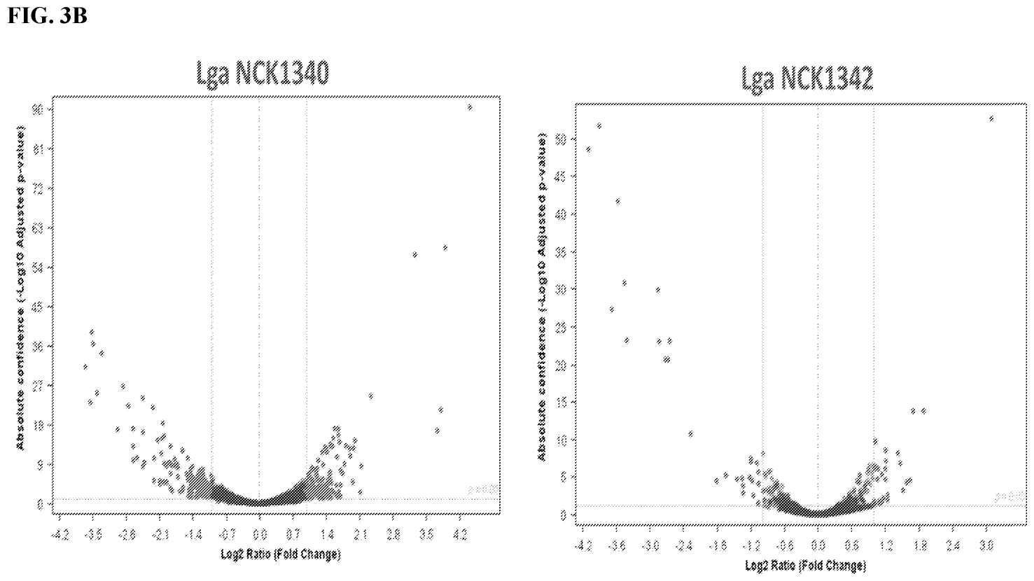

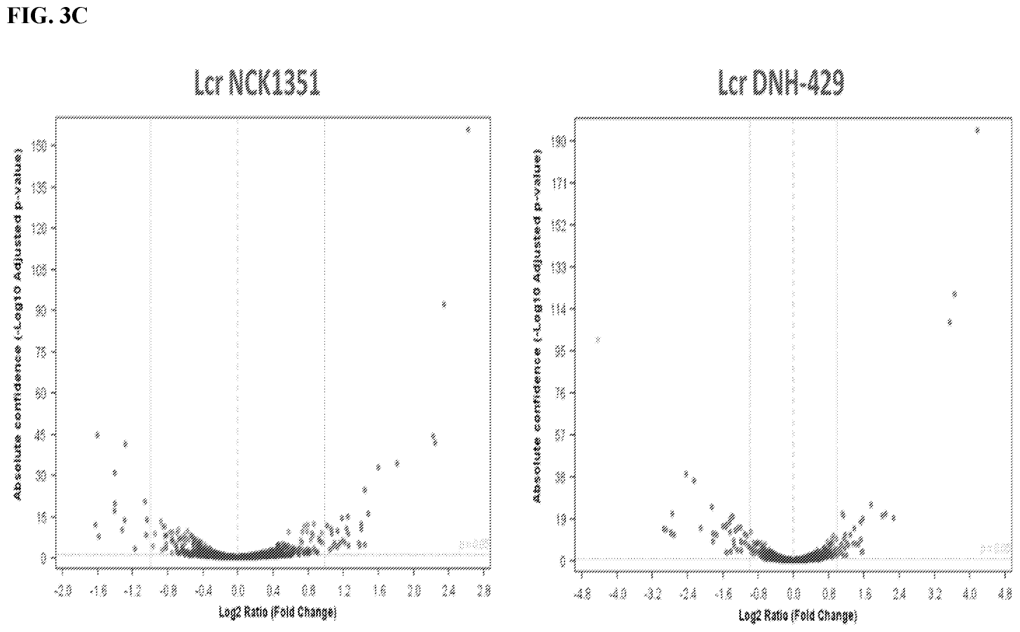

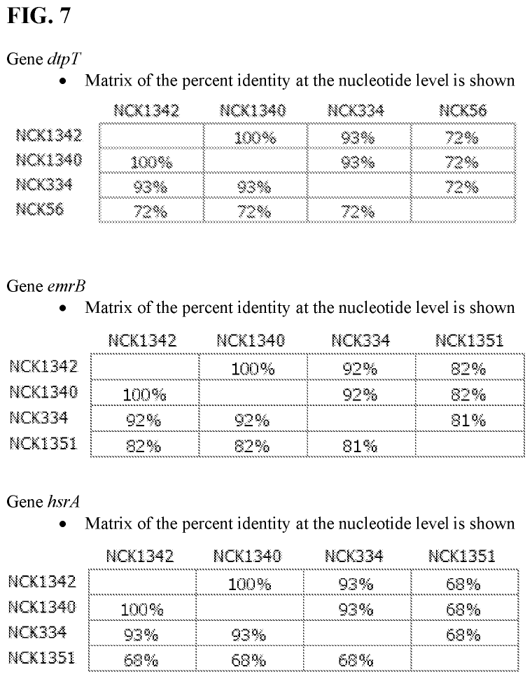

Overall, results indicated differential expression of a gene subset for each strain with the differential expression log₂ values ranging from 4.45 to −4.42. Genes encoding numerous types of transporters including phosphotransferase system (PTS) transporters, multidrug transporters, di-tripeptide transporters, and members of the major facilitator superfamily (MFS) were up-regulated. In addition, genes encoding glucosidases, permeases, hydrolases and hypothetical proteins were up-regulated in select bacteria grown in media comprising SDM with 0.5% glucose and pomegranate extract compared to control (SDM with 0.5% glucose). In particular, across species and strains, homologues of certain genes were upregulated, strongly indicating their involvement in exposure and/or growth of these strains in the presence of pomegranate extract. These genes are likely implicated in the adaptive response. For example, dtpT, a di-tripeptide transporter, was up-regulated in L. acidophilus NCFM, L. gasseri NCK334, NCK1340 and NCK1342 when grown in media containing pomegranate extract. A transporter annotated as a multidrug transporter, emrB, was the highest up-regulated gene in L. gasseri NCK1340 and L. crispatus DNH-429, and the second highest up-regulated gene in L. crispatus NCK1351 when grown in SDM containing the pomegranate extract compared to SDM without pomegranate extract. A second transport protein encoding gene, hsrA, was up-regulated in L. gasseri NCK334, NCK1340, NCK1342 and L. crispatus NCK1351 when grown in SDM containing the pomegranate extract compared to SDM without pomegranate extract. An overview of differential gene expression for each strain is outlined below, and volcano plots for each are shown in FIGS. 3A-3C, and details of select genes are noted in Table 2 and FIG. 7.

Lactobacillus acidophilus NCFM:

Genes that included higher expression in SDM with pomegranate extract compared to the control included a di-tripeptide transport protein (LBA1848 dtpT log₂ ratio 1.96), multi drug transporter proteins (LBA0552 log₂ ratio 2.23 and LBA1621 log₂ ratio 2.23), permeases (LBA1952 log₂ ratio 2.12) and a glycosidase (LBA1744 log₂ ratio 1.5, LBA0753 log₂ ratio 1.6). With the exception of LBA0753, these genes were not reported as differently expressed in a previous study looking at the ability of L. acidophilus NCFM to metabolize the dietary plant glucosides amygdalin, esculin and salicin (Theilmann et al. 2017, ‘Lactobacillus acidophilus Metabolizes Dietary Plant Glucosides and Externalizes Their Bioactive Phytochemicals’, MBio, 8.). The gene lba0753 was found to be up-regulated (log₂ 2.3) when grown on esculin compared to lactose. Down-regulated genes included an ABC transporter operon (LBA0151 to LBA0154, log₂ ratios −1.42 to −1.23), and a second operon that includes a prolyl aminopeptidase, response regulator, hypothetical, sensor and membrane proteins (LBA1658 to LBA1662, log₂ ratios −0.82 to −1.23).

Lactobacillus gasseri ATCC33323:

An ermB homologue annotated as a membrane transporter (LGAS_RS03570 log₂ ratio 4.45), in addition to two additional membrane transporter genes (LGAS_RS03610 log₂ ratio 1.9 and LGAS_RS06170 ratio 1.88), a 6-phospho-beta-glucosidase (LGAS_RS08205 log 2 ratio 1.86), a PTS beta-glucoside transporter subunit EIIBCA (LGAS_RS08210 log₂ ratio 1.34) and a PTS sugar transporter subunit IIC (LGAS_RS00955 log₂ ratio 1.35) showed higher expression in SDM with pomegranate extract compared to control. The majority of down-regulated genes encoded hypothetical proteins and a prophage encoded within the chromosome of L. gasseri ATCC33323.

Lactobacillus gasseri NCK1340:

Genes that included higher expression in SDM with pomegranate extract compared to control included transporter genes emrB (PROKKA_01205 log₂ ratio 4.4), hsrA (PROKKA_00450 log₂ ratio 2.23), dtpT (PROKKA_01295 log₂ ratio 2.15), yxdM (PROKKA_00450 log₂ ratio 1.8) and numerous hypothetical proteins. The majority of down-regulated genes encoded hypothetical proteins and bacteriocin related genes.

Lactobacillus gasseri NCK1342:

A smaller set of genes were up-regulated in L. gasseri NCK1342, but included the transport proteins hsrA (PROKKA_01897 log₂ ratio 3.10) and dtpT (PROKKA_00557 log₂ ratio 1.47). Similar to L. gasseri NCK1340, the majority of down-regulated genes encoded hypothetical proteins and bacteriocin related genes.

Lactobacillus crispatus NCK1351:

In the case of L. crispatus NCK1351, the three most highly up-regulated genes were a surface layer protein (PROKKA_01116 log₂ ratio 2.36), and the transporter genes hsrA (PROKKA_00077 log₂ ratio 2.25) and emrB (PROKKA_00476 log₂ ratio 2.35), which were both up-regulated in other strains discussed above. In addition, an operon of three hypothetical proteins and an amidohydrolase (PROKKA_01607 to PROKKA_01610, log₂ ratio 1.49 to 1.19) were up-regulated. Four genes from a second operon related to glutamine transport were up-regulated to a lesser extent (PROKKA_00420 to PROKKA_00420 log₂ ratio 1.16 to 1.10). Other genes of interest that were up-regulated included bglA, a Aryl-phospho-beta-D-glucosidase (PROKKA_00151 log₂ ratio 1.45), and a PTS system mannitol-specific EIICBA component (PROKKA_00646 log₂ ratio 1.22). Down-regulated genes included hypothetical proteins, and a glucose-specific phosphotransferase enzyme IIA component (PROKKA_01677 log₂-1.58.

Lactobacillus crispatus DNH-429:

Genes that were more highly expressed in SDM with pomegranate extract compared to control included hypothetical proteins (Peg.30 log₂ ratio 4.18, Peg85 log₂ ratio 3.55, Peg.1136 log₂ ratio 1.77 and Peg.1137 log₂ ratio 1.58), permeases (Peg.84 log₂ ratio 3.66, Peg.1366 log₂ ratio 1.53) and a glycosidase (Peg.55 log₂ ratio 1.3). Further analysis of the highest up-regulated gene (hypothetical protein Peg.30) showed that it contained conserved domains found in transporter proteins such as the drug resistance transporter emrB. Interestingly, ermB was the second highest up-regulated gene in L. crispatus NCK1351 and the highest up-regulated gene in L. gasseri NCK1340. Two glutamine transport genes were also induced in L. crispatus DNH-429, but to a lesser extent (log₂ ratios <1) than L. crispatus NCK1351. Similar to other strains, down-regulated genes included ABC transporters and hypothetical proteins.

### Example 4: Preparation of Pomegranate Extract and Media for Growth Curves

Preparation of pomegranate extract for growth curves was followed as described previously (Henning et al. 2017. Anaerobe, 43: 56-60). Pomegranate extract was dissolved in ultrapure water at a concentration of 7 mg/ml and vortexed for 10 min. The solution was then centrifuged at 4,000 rpm for 10 min and filter sterilized through at 0.45 μm filter. Aliquots of the stock solution were stored at −20° C. For growth curves, SDM was used as the base media (Kimmel and Roberts 1998. Int J Food Microbiol, 40: 87-92), with the addition of glucose at 1% for transfers, or 0.5% for growth assays. Stock pomegranate extract was diluted in SDM to 400, 800, or 1200 μg/ml for transfers and growth assays.

### Example 5: Growth of Lactobacilli in the Presence of Pomegranate Extract

Lactobacilli strains (FIG. 4) were inoculated from −80° C. glycerol stocks into MRS broth and grown overnight at 37° C. under ambient atmospheric conditions. Cultures were then used to inoculate SDM media (1% glucose, with or without pomegranate extract 400 μg/ml) for two overnight transfers prior to growth assays. Growth assays were performed in 96 microtiter well plates in control SDM (0.5% glucose) and SDM with 0.5% glucose and 400, 800 or 1200 μg/ml pomegranate extract. The OD 600 nm was recorded every hour over 24 to 48 hours using a microtiter plate reader (BMG Technologies). Plate assays were repeated at least twice.

### Example 6: Adaptation Experiment

Strains were inoculated from −80° C. glycerol stocks into MRS broth and grown overnight at 37° C. under ambient atmospheric conditions. Cultures were then inoculated (1%) into control SDM (1% glucose) and SDM with 1% glucose and 400 μg/ml pomegranate extract for an additional two transfers. Subsequently growth curves were performed in 96 well plates as described above. To determine loss of the adaptation effect, cells were transferred as follows: 1; four times in control SDM (1% glucose), 2; twice in control SDM (1% glucose) followed by two transfers in SDM with 1% glucose and 400 μg/ml pomegranate extract, 3; twice in SDM with 1% glucose and 400 μg/ml pomegranate extract followed by two transfers in control SDM (1% glucose) and 4; four times in SDM with 1% glucose and 400 μg/ml pomegranate extract. Growth curves were then performed in 96 well plates as described above. Plate assays were repeated at least twice.

### Example 7: RNA-Sequencing Data Analysis

Strains were selected for RNA-seq analysis based on the growth assays. Cells were transferred twice in SDM with 1% glucose and pomegranate extract 400 μg/ml then duplicate cultures were grown to mid-log phase (OD600=0.5-0.7) in control SDM (0.5% glucose) broth or SDM with 0.5% glucose and pomegranate extract 400 μg/ml at 37° C. under ambient atmospheric conditions. Cells were pelleted and flash frozen and pellets stored at −80° C. Methods for RNA isolation and RNA sequence analysis were as described previously (O'Flaherty and Klaenhammer 2016. Appl Environ Microbiol, 82: 6091-101.). Total RNA was extracted using the Zymo Direct-zol RNA MiniPrep kit (Zymo Research, Irvine, Calif.). DNA was removed by incubating samples with Turbo DNase as described by the manufacturer (Ambion Inc., Austin, Tex.), purified using the RNA clean and concentrator kit (Zymo Research), and checked for integrity by capillary electrophoresis on the Agilent Bioanalyzer (Agilent Technologies, Santa Clara, Calif.).

rRNA was removed using the Ribozero Bacteria kit (Illumina, San Diego, Calif.), followed by library preparation using the TruSeq Stranded RNA Sample Prep kit (Illumina, CA). Libraries were quantitated via qPCR and sequenced on one lane for 151 cycles from one end of the fragments on a HiSeq 4000 using a HiSeq 4000 sequencing kit version 1; reads were 150 nts in length. Fastq files were generated and de-multiplexed with the bcl2fastq v2.17.1.14 Conversion Software (Illumina). Adapter sequences were removed and raw sequences were assessed for quality using Fast QC version 0.11.4 (bioinformatics.babraham.ac.uk/projects/fastqc/). Subsequent processes were performed with Geneious (Kearse et al. 2012). Sequences were mapped to the relevant reference genomes using the Geneious mapper (Kearse et al. 2012). Geneious software was used to calculate the normalized transcripts per million (TPM), differential expression p-value, and differential expression absolute confidence to compare expression levels between the control and test conditions.

### Example 8: Preparation of Samples for Targeted Proteomics

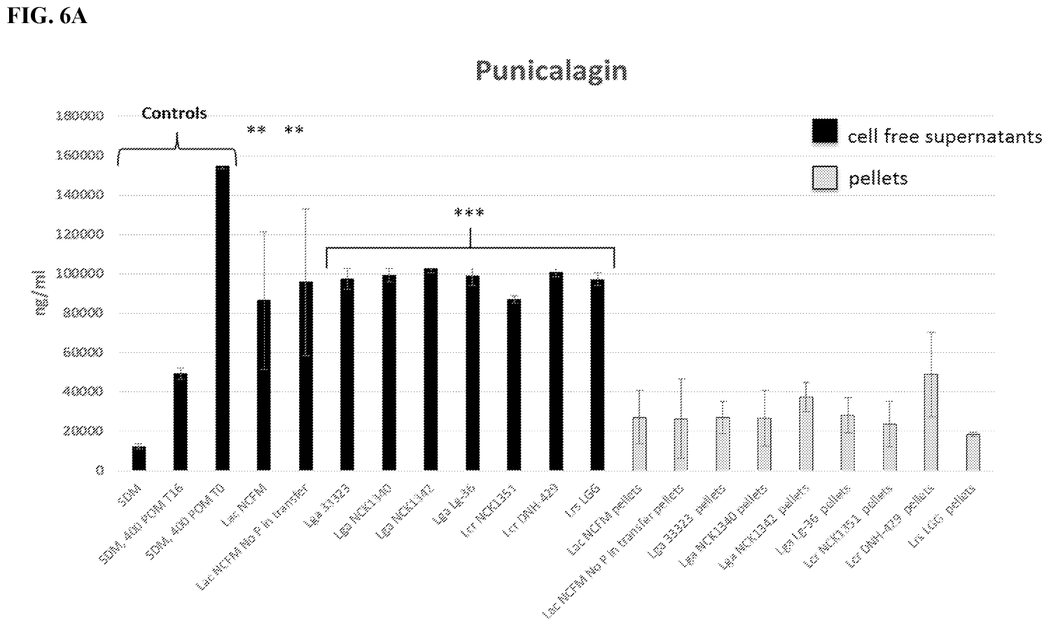

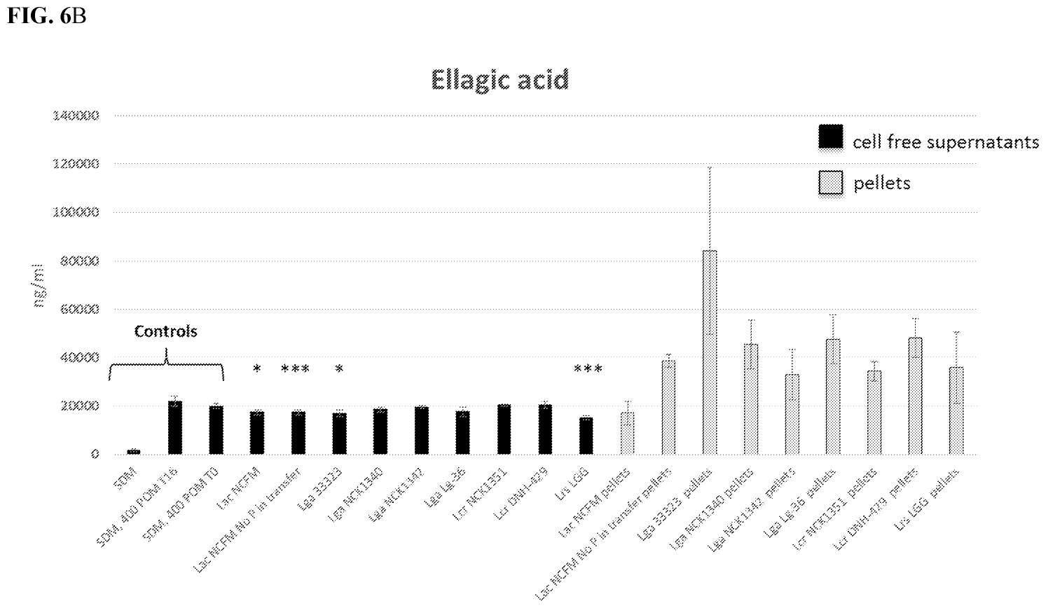

Strains with commercial potential and favorable growth assay phenotypes were selected for detection of punicalagin and ellagic acid. Cultures were transferred and grown as described above for growth curves and for the L. acidophilus NCFM adaptive response. After two transfers in in SDM in SDM (1% glucose with 400 μg/ml POM extract), cells were inoculated (1%) into 10 mls of SDM (0.5% glucose with 400 μg/ml POM extract) and grown for 16 hours at 37° C. After 16 hours cultures were centrifuged at 4,000 rpm for 10 minutes and 500 μl of cell free supernatant transferred to labelled cryogenic tubes for storage at −80° C. Preparation of the supernatant was performed to mimic the calibration curve for both punicalagin and ellagic acid. Forty μl of supernatant was diluted with 360 μl of water and ran on the triple quadrupole mass spectrometer. The pellets, from 500 μl of bacterial culture were resuspended in 250 μl of methanol, vortexed vigorously and centrifuged at 10,000×g for 15 min. Two hundred μl of the supernatant was removed and dried fully prior to being reconstituted in 100 μl of water. This 100 μl was placed into an LC vial and run on the triple quadrupole mass spectrometer. Results are shown in FIGS. 6A (punicalagin) and 6B (ellagic acid).

### Example 9. Adaptive Response Comparison

L. acidophilus NCFM (Lac56), L. plantarum Lp-115 and L. rhamnosus GG were grown in media comprising SDM with 0.5% glucose and SDM with 0.5% glucose and pomegranate extract 800 μg/ml.

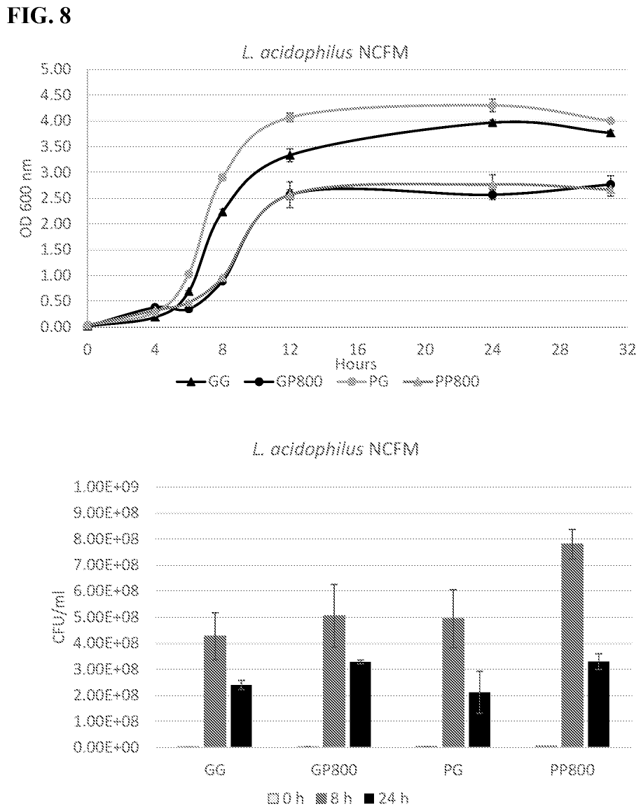

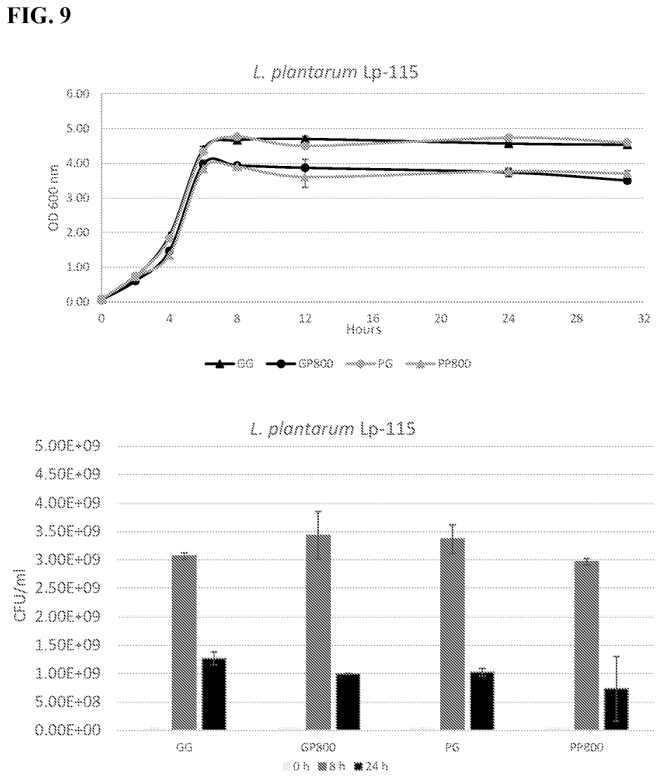

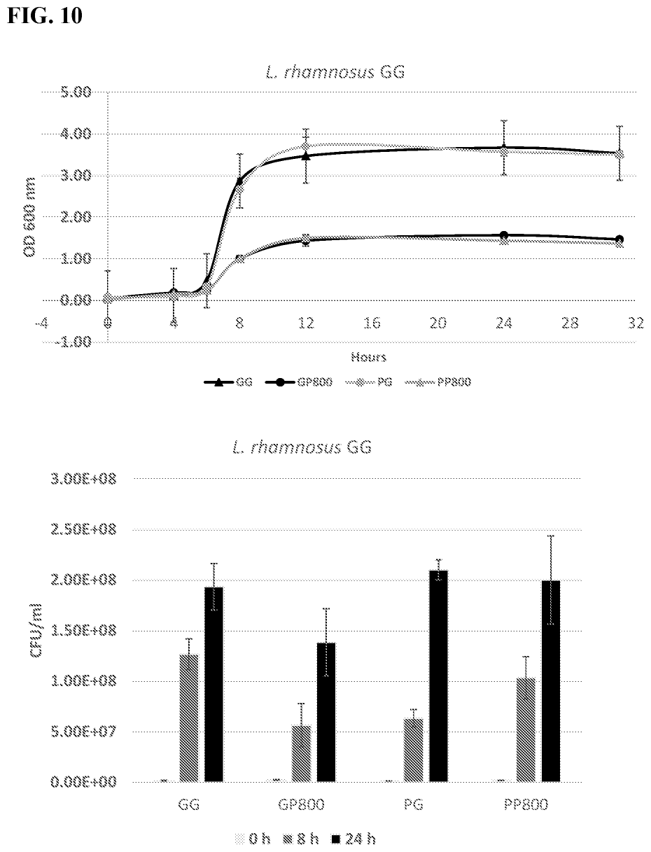

L. acidophilus NCFM, which is capable of an adaptive response when grown on pomegranate extract, is shown to have better growth in media with and without pomegranate extract when pre adapted in growth media comprising pomegranate extract when compared to bacteria that are not capable of the adaptive response, e.g., L. plantarum Lp-115 and L. rhamnosus GG. This better growth was observed whether or not the bacteria were previously adapted to growth in media comprising pomegranate extract. See FIGS. 8-10.

TABLE 1
Lactobacilli species tested

| Genus | Species | Number of strains |
|---|---|---|
| Lactobacillus | acidophilus | 7 |
| | brevis | 1 |
| | casei | 1 |
| | crispatus | 7 |
| | delbreckii subsp. bulgaricus | 1 |
| | fermentum | 1 |
| | gasseri | 22 |
| | johnsonii | 1 |
| | paracasei | 1 |
| | pentosus | 1 |
| | plantarum | 3 |
| | reuteri | 1 |
| | rhamnosus | 2 |
| | salivarius | 1 |

TABLE 2
Details of genes

| | | | | Length |
|---|---|---|---|---|
| Species | Strain | Locus | Gene annotation | bp |
| dtpT and homologs | | | | |
| Lactobacillus acidophilus | NK56 | LBA1848 | dtpT | 1494 |
| Lactobacillus gasseri | NCK1340 | PROKKA_01295 | dtpT | 1494 |
| Lactobacillus gasseri | NCK1342 | PROKKA_00557 | dtpT | 1494 |
| Lactobacillus gasseri | NCK334 | LGAS_RS02570 | peptide MFS transporter | 1494 |
| ermB and homologs | | | | |
| Lactobacillus gasseri | NCK1340 | PROKKA_01205 | emrB | 1473 |
| Lactobacillus gasseri | NCK1342 | PROKKA_00047 | emrB | 1473 |
| Lactobacillus gasseri | NCK334 | LGAS_RS03570 | MFS transporter | 1473 |
| Lactobacillus crispatus | NCK1351 | PROKKA_00476 | emrB | 1473 |
| hsrA and homologs | | | | |
| Lactobacillus gasseri | NCK1340 | PROKKA_00450 | hsrA | 1383 |
| Lactobacillus gasseri | NCK1342 | PROKKA_01897 | hsrA | 1383 |
| Lactobacillus gasseri | NCK334 | LGAS_RS06170 | MFS transporter | 1394 |
| Lactobacillus crispatus | NCK1351 | PROKKA_00077 | hsrA | 1374 |

SEQUENCES
dtpT from L. acidophilus NCK56
SEQ ID NO: 1
TTGGGTAAACGAGATACAAATACAGCATTTTTCGGACAACCTAAGGGTTT
GTCTACATTATTCTTTACTGAAATGTGGGAGCGTTTCAGTTACTACGGCA
TGCGAGCAATTCTTTTGTTCTACATGTACTATGCAGTTACAAAGGGCGGT
TTAGGAATGGATCAAACTACCGCTGCATCGGTGATGTCGATCTACGGCTC
GCTTGTTTATTTGTCAGCTTTAGTAGGTGGCTGGCTTTCCGATAGAGTAT
GGGGATCAAGAAAGACCGTCTTTTATGGTGGTGTATTGATCATGCTAGGT
CACATCGTGCTGGCTTTACCAGCGGGTGTAGGTGCATTATATAGTTCTAT
TGCTTTAATTGTGGTTGGTACAGGTTTATTAAAACCTAATGTATCAGATA
TGGTAGGGGGACTTTACTCCGTAGAAGATCGTCGTCGTGATGCCGGGTTT
AGTATTTTTGTTTTTGGTATTAACCTGGGTTCTTTTATCGCACCATGGCT
TGTTCCTTGGGCAGCTGAAGGATTCGGCTTCCATGCTTTTGGAAATGAAT
TGAACTTTCATGCTGGATTTTCGCTAGCTGCAATTGGAATGTTCTTTGGA
TTAGTTCAATATGTTTTAGGTGGTAAAAAATATTTGCCCACAAGTAGTCT
AGAACCTAATGATCCAATCGATAGAGGTGATTTGCTTAATATTATTAAGT
GGGTTGTTATTATCGTAGTTGCAGTTGCTGCAATTTTAGCTGCATTAGCT
GGATTTGGTCAATTGAACGTTGATAATATAATTACTATGTTGACTGTTTT
AGCAATTGCATTGCCAATTTATTACTTCGTAATGATGCTACGCAGTTCAA
AAGTTACTAAAGTTGAACGTTCACGTGTTAAAGCTTATATTCCATTATTC
TTTGCGGCAGCTATTTTCTGGGGAATAGAAGAATCAGGATCTGTTGTTTT
AGCTTTGTTTGCAGAGCAAAGAACTGTACTTCATGTTGGTAATTGGCATT
TTACAGCAGCCAACTTCCAATCACTTAATCCTTTGTTTATTATGATTTTG
ACGCCAGTTTTTGTATGGCTATGGGAACACTGGAAGAAACAACCAAGTGC
GCCGGGTAAATTTGCTGCTGGACTTGTTTTTGCCGGTTTATCTTATATGT
GGATGGCTTTGCCAGGGATGCTTTATGGTACAAGCGGTCGAGTTAGTCCT
TTCTGGCTAGTTGGTTCATGGTTCATTGTTGAAATTGCCGAAATGTTGAT
TTCACCTATTGGTTTATCAATTACGACTAAGCTTGCGCCAAATGCATATC
GTTCACAAATGATGAGTATGTGGTTTATGGCTGATGCGACGGGTCAAGCA
ATCAATGCACAAATTGTTAAGTTCTATTCCACTGGTACAGAAGTACAATA
CTTTACTGTCATTGGATTAGTTAGTGTAGTCTTCGGTATTATTATGTTCT
TATTAGTTAAGAAGATTAATGTATTGATGGAAGGCGTACATTAA.
emrB from L. gasseri NCK1340 and NCK1342
SEQ ID NO: 2
ATGGATCATCAACCAACCGATATTCACGGAAAAGCCTATAATCGAAACTT
GCTTGTTCTAGTTTTAATTATTGGATCTTTCTGTACTGTGTTAAATGGAA
CCTTGCTTGCAACAGCTCTTCCCTCAATTATGCGTTCCTTTCATATCAGT
ACAGCAACTGCTGAATGGCTTTCAACTGCATTTTTATTAGTTAATGGAGT
CATGATTCCAGTTTCAGCTTGGCTAATTAATCGTTTTGGCTCAAGAAAAA
TGTATTTGACAGCAATGTCAGTTTTCTTTATTGGAACAGTGACTGCTGCA
GTTGCGCCAAATTTTGGGACTTTATTAACTGGAAGAATTATTCAAGGACT
CGGCGTGGGTGTTACTATGCCGCTTCTTCAAACCATCATGCTTTCAATCT
TTCCCGCTAATAAACGCGGTGCTGCGATGGGAACAGTTGGTATTGTTATT
GGTCTCGCACCAGCTATTGGTCCAACCTTATCTGGGTGGATTGTTGATAA
TCTTTCCTGGCGTTACTTATTCAGTATTATTGCTCCAATTGCTGGAATTG
TTATTATTTTAGCGTTTTTCCTAATTAAAGACGTTCTTCCAACTAAGAAA
GAAAAAATTGACGTTTGGTCAGTGACAACTTCAACTATTGGTTTTGGTAG
TTTACTTTACGGCTTTTCAGAAGCTGGTAATAAAGGATGGACTAACCCTG
AAATTCTAGGATTTATTGGCGTAGGAATTATATTTGTTATTCTTTTTGGC
ATGCGGCAATTAAAGATGGCAGATCCATTTTTAGATATTACCGTCTTCAA
ACATTTCGAATTTTCTCTAGCAGCTGCTTTAAGTGGTATTACCAATTTAG
CAATGGTTGGTATTGAAATGGTTTTGCCATTATATATTCAAAATTTACGT
GGTGTATCAGCATTTCATTCAGGATTAATGCTATTACCTGGTGCCTTGAT
GATCGGAATTATGTCACCAATTACGGGACGTTTATTCGATAAATATGGTG
CACGAAAAATGGCAATTACTGGAATGACCCTCTTAACACTTGGCACTATT
CCATTTGTATTCTTAACTGCAGAAAGCTCATATACTATGATTATTGTTTT
GTACGCAATTAGAATGGTCGGTGTTGCTTTAGTTATGATGAACGTTACCA
CTTCAGGAATGAACTCATTGCCTTTAAACAAAATCTCCCACGGAACAGCA
GTGAACAATACTTTTCGACAAGTTTTAAGTTCAATTGGTACTGCTATTCT
CGTTTCAATTTTAACTACTACAACCAACAACCATATGCCAGCAAAAGAAC
TGCTTCATACCCTTCCACTTCAATATAAGAATCAAGCAATCAATGCAACT
TTAGATGGATTCCGTGCTTCATTTGCAATGAGTATTCTTTTTGCCTTAGT
TGCTTTAGTTCTTTCTTTCTTCTTAAAGAAAGGCAACCGTGCACGTGAAA
ATCAAGAAGAGGTGAACGGATAA.
hsrA from L. gasseri NCK1340 and NCK1342
SEQ ID NO: 3
ATGAAAGTACAAACTAAAAATACACTTGCAGTTTTGGCAACAGCATTCAT
GTCTTTTGTTGGAATTTTAACTGAAACTAGTTTAAATGTAACTTTTCCTG
CTATGATGAAGCAATTCCAAGTTTCATTGGACACTATTCAATGGATTACA
ACTGGCTATTTGCTGATGATTGCGATTATTATGATTAGCTCTTCTTATCA
AAATGAACGATTTACAGCAAGACAATTGTTCATTTCATCAGCAGTTGCCT
TTATAACTGGAAGCATGATTTCTGCTTTTGCATCAAGTTTCTACATTTTG
CTTTTAGGAAGATTAATTTCAGCTCTGGGAGTAGGTCTATGCACTCCGAT
GATGTTTAATTTAATCGTGGAGGTAATGCCTAGGCAAAGCTGGGGATTTT
ACATGGGAATTGCTGGTTTAGTAATTGCAATGGCACCGACTTTAGGTCCT
ACATTTGGAGGCAGTGTTTCGTATTATCTTAATTGGCGCTGGATTTTCAT
TATTGCTGCAATTTTTGCCCTCTTTGTCTTTATTTCCGGTATTTTTGTAA
TTGGAAGTTATCATCCGGTTCAAAAGAAGTCATTTAACTGGCTAGCTTAT
ATATTTTTAAGTCTTAGTTTAGTAAGCTTAGTTATTGGTGTAAATCAATT
AAGTAAAGGATTACAAAACTGGCGTCTTTGGGGACTATTAATCTTAACGG
TTGTTTTGTTCATTTTATTTGTTACAATTTCAAAAAGATCTACTCGAAAG
TTGCTTGACTTGAAAGTTTTTGAAGATAAAGCTTTCGTTTTTGGGGCATT
TGCGTACTTTTTGCTTCAATTTATCAATATTGGTGTAAGTTTTGTATTAC
CCAATTATATTCAAATTGTTAACAAACAAAGCTCTTTAATCGGTGGTCTG
GTTTTACTTCCAGGAAGTATTATTGCTGGACTTTTAAATCCGTATTTTGG
CCGTTTATATGATCGCTTAGGTGCAAGAGTGCCATTATATGGTGGAGCTT
TTTTAATGGCATTAGGGAGCTTCTTATTGGCTAATTGGGGACTAAACTTA
AATACCTGGATGATTATCTGCTTTTATGGAATTTTGATGTTAGGACATCG
AATGTCGTTCAGTAATACAATGGCCCAAAGCTTGAAAATAGTAGATAATA
ACCTTAAGTCAGACGCAACTGCTGTTTGTCAGACAGCGCAACAACTAGCT
GGTTCAATGGGAACAGCAATTTTAGCAGCAATTATAGCTATTTTTCAGAA
TAAGCATACAGCAAAATACACTACTTTGACTGCTCAGGGAAGTATGGCAG
CATTTTATTTCACTTTTGGCCTTGGAATTTTGATTTTAATATGTGATTGG
ATTATGTTTAAGCTGAGTAAAGAAAATAAGTAA.
LBA1744 (glycosidase) from L. acidophilus NCK56
SEQ ID NO: 4
TTGATTTTCAAACGTACTTTGGTTAAATACACAGCAGCTTTATCAATTTT
CTTTACAGGTCTTTCAGCAGTTAGCGTTCCAGCAACTGTTCACGCTGACG
ATGTTGAAAATACCACTACTGTTGACACCAACAACTCAGCTTCATCTATT
GAAACCAGTACACCTGAATCAAATTCAGTAAAGAAGGTTTCAGCTACAAC
CCAAAAGCGTAACGCTATTGTTAAGCTTGCTAAGAAGCAAGTTGGTAAGC
CTTACGTTTGGGGTGCTACTGGCCCTTACGGATTCGACTGCTCAGGCTTA
ACTACTTATGTTTACAAGAATGCAGCTAACAAGACTTTACCAAGAACTAC
TTACGGTCAAATTACTATCGGTAAATCAGTATCAGTTTCAACTAAATCTC
TTAAGAAGGGTGACTTATTGTTCTGGGGTAACTCACACGTAGGTATCTAC
ATTGGTAACGGTAAGTTTGTTCACGCTCCAGCTCCAGGTCAAAATGTTAA
GACTCAAACTTTAGCATCATTCTACCCATCAGCTGCTAAGCGTGTTATCG
GTTAA.
LGAS_RS08205 (6-phospho-beta-glucosidase)
from L. gasseri NCK334
SEQ ID NO: 5
ATGTCATTTCCAAAGAACTTTTTATGGGGCGGAGCTACTGCCGCAAATCA
AATTGAAGGTGCGTATGATGAAGATGGTAAGGGATTATCAGTCACTGATA
TTACAACTGCTGGTAGTTTAGATGCGCCAAGAATGTTGACTTACAAGTTG
AATGGCAAATTAGAAAAAACGCCAGCCATTCCCGGTGCGGGCTTGCCAGA
AGGTGCAGTTGGTGCAATTGATCCAAATGAATATTATCCAAACCACGTGG
CAATTGATTTTTACCACCACTATAAAGAAGACATTAAGATGTTTGCTGAA
ATGGGCTTTAAGACCTTCCGCCTTTCAATTGCCTGGACTAGAATTTTCCC
TAAAGGCGATGAAGAAAAGCCAAATCAAGCTGGATTGGATTTTTATCGCC
ATGTATTTGAGGAATGTAAAAAGTACGGGATTGAACCTTTAGTTACTATT
TCTCACTACGAAGATCCACTATACCTCAGTGAAAAATACCATGACTGGGG
CGATCGAAAAATGATCGATATGTATGTTAAATACGCAACTACTTTGTTTG
AAGAATACAGAGGACTAGTTAAGTACTGGCTTACATTTAATGAAATTAAT
TCAACGCTTTTAATGCTTAGTTCATTTGGTAATAATGTAACTGATGATAA
GGTATATCAACATGCCTACCAAAAACTGCACTACCAATTTGTCGCTAGTG
CTCGCGCAGTTAAGATTGCTCATGCATTAGATCCTAACTACGTAGTTGGT
AATATGATTTGTGGTATTGTGGACTACCCATTAACACCAGATCCTAAAGA
TATTTTGGCTAACCGTCATATGTGGGAACAAAATATCTTCTACTGCGGCG
ATGTGCAAGCAAAAGGTAAGTACCCAACTTATGCTAAGCGTTTATGGAAT
GAACATAATGTTCATTTAGATATTACTGAGCAAGATCGAAAAGATTTGCT
TGAAGGGAAAGTTGATATTTATACCTTCTCTTACTATATGTCTAACATTA
TTACTACTCATGAAGTAAAGGATAAAGTTGGCGGCAATTTTGCTGCTGGT
GTTAAGAACCCATACTTGAAGTATTCTGAATGGGGCTGGGCAACTGATCC
AGATGGTCTTCAATATTATCTTGAAGTAATGTATGATCGCTACGATATTC
CAATGATGGTTGTTGAAAATGGTCTTGGTGCAGTTGATAAGATCAGTGAT
GATGGTAAGATTCATGATGATTACCGCATTGATTACTTGAGAATGCACAT
TAAGGCAATGGAGCGTGCAATTGATGATGGTGTAGATTTAATTGGTTACA
CTACTTGGGGCTGTATTGACTTAGTTTCTGCAGGTACTGGTCAAATGAGT
AAACGTTACGGCTTTATCTATGTTGACAGAGACGATGAAGGTAAGGGTAC
TTTGAAGAGAATGCCGAAAGATTCATTCTATTGGTACAAGAAAGTTATTG
AATCTGATGGTAAAGATTTAGACTAA.
PROKKA_01116 (surface layer protein slpH3)
from L. crispatus NCK1351
SEQ ID NO: 6
ATGAAGAAAAATTTAAGAATTGTTAGCGCTGCTGCTGCTGCTTTATTAGC
TGTTGCTCCTGTCGCTGCTTCAGCTGTTTCTGTTAACGCTGCAAGCTCAA
GTGCTGTTCAAACTGCTACCAACATTGGTACTGTTTTACCATTAACTGAT
GGTTCTACTGTTAACGTTAAGCCAAACATTTCATTGAACACTTCAGCATA
CGAAGGTGTTAAGGCAAACATTTCAGTATCATTCTCAGCAACTGTTGACG
GTACTACTGCTACCTCTAACTTCACTCCAAATGCTTCAACTATTGAACTT
TGGAAGAATGAAAAGGATAAGGTTACCCAAGTAACTGATTTACAACGAGT
AACTTCATCAAACGCTGGTGCTACTTACCAAGTTAAGATGACTCAAGTTG
GCTTGAACTTCGGTTCACAAAACGCTAACAAGAAGGTTACTTTGACTTTC
CCTGAGGGTGACATGTTCAAGACCGCTGATACTTCTTTAGCACAATCACA
CGAAGTAAAATTAGACCAAAACGGTACTATTACTTTGCCAGAAGTAGTTA
TGAACGTAACTGCTAAGGACTTCGCTAACCCAGCAGTAGTTAACTGGTAC
AACACTGCTACTAACGCAGTTGTAAGTACTGGTAACATTGAACTTTTCGC
AGGTTCAGATGCTGGTAAGATGAACGTTGCTCAAGTTGTTTCAGCAACTG
AAAAGAAGTACCACGCAAGCAACTACGGTACTAAAGCTAACCAAGAATCA
AGCACTATTTCATACACCAACAACCTTAAGGATGCTTTAAAGGCTATGAA
CGTTGATGTTGATGCTCAAGGCTGGTTCGTTGCTCCTAAGTCATTCACTT
TCAACATGACTGCTAAAGCTAACAACAATGATGCTTCAAGTACCTTAGCT
GTAACTGTTTCAGTTCCAAACGGTAAGGACATGACTGTACCAAGCCAAAG
CAAGACTGTTATGCACAACGCATTCTTCTATGACAAGAACGGCAAGCGTG
TTGGTTCTGACAAGGTAACTCGTTACAACTCAGCAACTGTTGCTATGAAT
ACTACTACTATCAACGGCAAGGCTTACTACGAAGTAATCGAAAACGGTAA
GGCTACTGGTAAGTTCATCAACGCTGCCAACATTGATGGTACTAAGCGTA
CTTTGAAGCATAACGCATACGTTTACAAGTCTTCAAAGAAGCGTGCTAAC
AAGGTTGTTCTTAAGAAGGGTACTGAAGTAGTTACCTACGGTGGTGCTTA
CACCTTCAAGAACGGCAAGCAATACTACAAGATCGGTAACAATACTGACA
AGACTTACGTAAAGGTTTCAAACTTTTAA.
PROKKA_00151 (bglA a Aryl-phospho-beta-D-
glucosidase) from L. crispatus NCK1351
SEQ ID NO: 7
ATGCAATATGTCAAAAATCATGGCATTCAAATGGACATTACAGATGAAGA
TAAAGAAATTATGAAGAAAGCTAAAGCTGATTTCCTAGCATTTTCTTACT
ATTCATCCTGGGTTTTATCTAGCGATAAAATTCCTGATGGTGAAGCTCCT
AACCATTACCTTAACAAGGGCGGAGTTGAGTCCAAATATGTTAAGACAAA
CGATTGGGGCTGGGCAATTGATCCACTAGGATTTAGGAATGCAATTACTA
CAATGTACAACTACTACCGTATTCCTATTTTCCCTATTGAAAATGGTATC
GGCCTCAAGGAAACTTGGGACGGTGAGCATATGATTGAAGATGATGAGCG
TATTGCTTATCACCGCGACCATATCAAAGCAATGAAAGATGCTATTTTTG
ATGAAGGCGCCAAGGTGCTTGGTTATCTTGGCTGGGGATTAATTGATATT
CCAAGTTCTCATGCTGATATGGAAAAACGCTACGGTGCTGTTTACGTCAA
CAGATCAAACCACGATTTAAAAGATTTAAAACGTGTGCCTAAGAAATCAT
TCTATTGGTTTCAAAAGGTACTTAAAGATAATGGAGATGAATTATAA.

The foregoing is illustrative of the present invention, and is not to be construed as limiting thereof. The invention is defined by the following claims, with equivalents of the claims to be included therein.

That which is claimed is:

1. A probiotic composition comprising:
a probiotic bacterium capable of an adaptive response, wherein the probiotic bacterium has been pre-cultured in a culture medium comprising pomegranate extract; and
pomegranate extract.

2. The probiotic composition of claim 1, further comprising a prebiotic plant glycoside, wherein the probiotic bacterium is capable of converting the prebiotic plant glycoside into a bioactive aglycone.

3. The probiotic composition of claim 2, wherein the prebiotic plant glycoside is a punicalagin.

4. The probiotic composition of claim 2, wherein the bioactive aglycone is a urolithin.

5. The probiotic composition of any one of the preceding claims, further comprising a pharmaceutically acceptable carrier.

6. The probiotic composition of any one of the preceding claims, wherein the probiotic bacterium is adapted to growth in a medium comprising pomegranate extract (e.g., is a pomegranate-adapted bacterium, e.g., exhibits an adaptive response; e.g., has a reduced time to lag phase and/or an increased final optical density and/or increased tolerance to pomegranate extract in the growth medium as compared to a control).

7. The probiotic composition of claim 6, wherein the probiotic bacterium has increased expression of transporter genes and/or glucosidase genes, optionally dtpT, emrB, and/or hsrA.

8. The probiotic composition of any one of the preceding claims, wherein the probiotic bacterium is a human intestinal bacterial species.

9. The probiotic composition of any one of the preceding claims, wherein the probiotic bacterium is from the genus Lactobacillus or Bifidobacterium.

10. The probiotic composition of any of the preceding claims, wherein the probiotic bacterium is L. acidophilus, L. casei, L. paracasei, L. crispatus, L. gasseri, L. plantarum, L. rhamnosus, L. salivarius, L. fermentum, L. reuteri, L. johnsonii, B. longum, B. lactis, B. infantis, or any combination thereof.

11. The probiotic composition of any of the preceding claims, wherein the probiotic bacterium is L. acidophilus NCFM, L. acidophilus La-14, L. casei Lc11, L. crispatus NCK 1351, L. crispatus DNH-429, L. gasseri ATCC 33323, L. gasseri NCK 1138, L. gasseri NCK 1340, L. gasseri NCK 1341, L. gasseri NCK 1342, L. gasseri NCK 1343, L. gasseri Lg-36, L. plantarum Lp-115, L. plantarum Lpc-37, L. rhamnosus GG, L. salivarius Ls-33, or any combination thereof.

12. The probiotic composition of any of the preceding claims, wherein the amount of probiotic bacteria in the composition is about 10⁸ cfu/g to about 10¹³ cfu/g.

13. The probiotic composition of any of the preceding claims, wherein the amount of pomegranate extract in the composition is about 50 μg/ml to about 2000 μg/ml.

14. The probiotic composition of any of the preceding claims, wherein the pomegranate extract comprises a polyphenol content of about 55% to about 65% gallic acid equivalents.

15. The probiotic composition of any of the preceding claims, wherein the pomegranate extract comprises gallic acid, ellagic acid, and glucose.

16. The probiotic composition of any of the preceding claims, wherein the pomegranate extract comprises ellagitannins (e.g., punicalagins and/or punicalins), free ellagic acid and anthocyanins.

17. The probiotic composition of any one of the preceding claims, wherein the pomegranate extract is a powder and further comprises ash, sugars, organic acids, and nitrogen, and optionally a moisture content of less than 10%.

18. The probiotic composition of any of the preceding claims, wherein the probiotic composition is a dosage form of a tablet, a pellet, a hard capsule, a soft capsule, an emulsion, a powder, a dispersible powder, a lozenge, a troche, a chew, a gel, an aqueous or oily suspension, a spray, a granule, a suppository, a solution, a syrup, and/or an elixir.

19. A sachet comprising the probiotic composition of any of the preceding claims.

20. A nutritional supplement comprising the probiotic composition of any of the preceding claims.

21. The nutritional supplement of claim 20, wherein the nutritional supplement is a beverage, a nutritional bar, a fermented dairy product, a non-fermented dairy product, or any combination thereof.

22. A method of selecting a bacterium capable of an adaptive response, comprising:
(a) pre-culturing a bacterium in a culture medium comprising pomegranate extract to produce a pre-cultured bacterial population;
(b) culturing at least a portion of the pre-cultured bacterial population in a culture medium comprising pomegranate extract and determining if the pre-cultured bacterial population has a shorter time to lag phase and/or increased final optical density (OD) and/or increased tolerance to pomegranate extract as compared to a control (e.g., the same bacterium cultured in a culture medium comprising pomegranate but that was not pre-cultured in the presence of pomegranate extract and/or the same bacterium that was pre-cultured in the presence of pomegranate extract and then cultured in a culture medium without pomegranate extract); and
(c) selecting the bacterium used to produce the bacterial population that exhibits a shorter time to lag phase, and/or an increased final OD, and/or an increased tolerance to pomegranate extract as compared to the control, thereby selecting the bacterium (e.g., bacterial strain) capable of an adaptive response.

23. A bacterium capable of an adaptive response selected by the method of claim 22.

24. A method of adapting a bacterium capable of an adaptive response, comprising:
(a) pre-culturing a bacterium in a culture medium comprising pomegranate extract to produce a pre-cultured bacterial population;
(b) culturing at least a portion of the pre-cultured bacterial population in a culture medium comprising pomegranate extract, wherein the pre-cultured bacterial population exhibits a shorter time to lag phase and/or increased final optical density (OD) and/or increased tolerance to pomegranate extract as compared to a control (e.g., the same bacterium cultured in a culture medium comprising pomegranate but that was not pre-cultured in the presence of pomegranate extract and/or the same bacterium that was pre-cultured in the presence of pomegranate extract and then cultured in a culture medium without pomegranate extract).

25. A pomegranate-adapted bacterium adapted by the method of claim 24.

26. A method of increasing tolerance of a bacterial population to pomegranate extract in a culture medium, comprising culturing at least a portion of a bacterial population capable of an adaptive response in a culture medium comprising pomegranate extract to produce a pomegranate-adapted bacterial population, wherein the bacterial population capable of an adaptive response has been pre-cultured in a culture medium comprising pomegranate, thereby increasing the tolerance of the bacterial population to the pomegranate extract in the culture medium as compared to a control.

27. A method of producing a bacterial population having increased tolerance to pomegranate extract in a culture medium, comprising culturing at least a portion of a bacterial population capable of an adaptive response in a culture medium comprising pomegranate extract, wherein the bacterial population capable of an adaptive response has been pre-cultured in a culture medium comprising pomegranate, thereby producing a pomegranate-adapted bacterial population having increased tolerance to pomegranate extract in the culture medium as compared to a control.

28. A method of decreasing time to lag phase of a bacterial population when cultured in a medium with or without pomegranate extract, comprising culturing at least a portion of a bacterial population capable of an adaptive response in a culture medium comprising pomegranate extract to produce a pomegranate-adapted bacterial population, wherein the bacterial population capable of an adaptive response has been pre-cultured in a culture medium comprising pomegranate, thereby decreasing the time to lag phase of the bacterial population when cultured in the medium with or without pomegranate extract as compared to a control.

29. A method of producing a bacterial population having decreased time to lag phase when grown in a culture medium with or without pomegranate extract, comprising culturing at least a portion of a bacterial population capable of an adaptive response in a culture medium comprising pomegranate extract, wherein the bacterial population capable of an adaptive response has been pre-cultured in a culture medium comprising pomegranate, thereby producing a pomegranate-adapted bacterial population having decreased time to lag phase when grown in the culture medium with or without pomegranate extract as compared to a control.

30. A method of increasing final optical density (OD) of a bacterial population when cultured in a medium with or without pomegranate extract, comprising culturing at least a portion of a bacterial population capable of an adaptive response in a culture medium comprising pomegranate extract to produce a pomegranate-adapted bacterial population, wherein the bacterial population capable of an adaptive response has been pre-cultured in a culture medium comprising pomegranate, thereby increasing the final OD of the bacterial population when cultured in the medium with or without pomegranate extract as compared to a control.

31. A method of producing a bacterial population having an increased final optical density (OD) when grown in a culture medium with or without pomegranate extract, comprising culturing at least a portion of a bacterial population capable of an adaptive response in a culture medium comprising pomegranate extract, wherein the bacterial population capable of an adaptive response has been pre-cultured in a culture medium comprising pomegranate, thereby producing a bacterial population having increased final OD when grown in the culture medium with or without pomegranate extract as compared to a control.

32. A method of producing a bacterial population with a reduced fermentation time to achieve a final optical density (OD), comprising culturing at least a portion of a bacterial population capable of an adaptive response in a culture medium comprising pomegranate extract to produce a bacterial population having reduced fermentation time to achieve a final OD compared to a control, wherein the bacterial population capable of an adaptive response has been pre-cultured in a culture medium comprising pomegranate.

33. The method of claim 32, further comprising
culturing at least a portion of the bacterial population having reduced fermentation time in an additional culture medium with or without pomegranate extract.

34. The method of claim 32 or claim 33, further comprising
collecting the bacterial population having reduced fermentation time for the manufacture of a probiotic product.

35. A method of producing a probiotic bacterial population for the manufacture of a probiotic product, comprising culturing at least a portion of the pre-cultured probiotic bacterial population capable of an adaptive response in the presence of pomegranate extract to produce a probiotic pomegranate-adapted bacterial population, wherein the probiotic bacterial population capable of an adaptive response has been pre-cultured in a culture medium comprising pomegranate.

36. The method of claim 35, further comprising
culturing at least a portion of the probiotic pomegranate-adapted bacterial population in an additional culture medium with or without pomegranate extract.

37. The method of claim 35 or 36, further comprising
collecting the probiotic pomegranate-adapted bacterial population for the manufacture of a probiotic product.

38. The method of any one of claims 26 to 37, wherein the adaptive response is maintained by growth in a medium comprising pomegranate extract.

39. The method of any one of claims 22, 24, or 26 to 38, wherein culturing at least a portion comprises transferring the at least a portion to the culture medium with or without pomegranate extract.

40. The method of claim 39, wherein when transferring to a medium without pomegranate extract, the transferring is directly from a medium comprising pomegranate extract (e.g., no intervening transfers to media without pomegranate extract).

41. The method of any one of claims 22, 24 or 26 to 40, wherein the culture medium further comprises a carbohydrate.

42. The method of claim 41, wherein the carbohydrate is present in the medium in an amount from at least about 0.25% to about 3%.

43. The method of claim 41 or 42, wherein the carbohydrate is glucose, lactose, sucrose, fructose, galactose, trehalose, raffinose, or a fructooligosaccharide, or any combination thereof.

44. The method of any one of claims 22, 24, or 26 to 43, wherein the culture medium comprises about 50 μg/ml to about 2000 μg/ml pomegranate extract.

45. The method of any one of claims 22, 24, or 26 to 44, wherein the pomegranate extract comprises a polyphenol content of about 55% to about 65% gallic acid equivalents.

46. The method of any one of claims 22, 24, or 26 to 45, wherein the pomegranate extract comprises gallic acid, ellagic acid, and/or glucose.

47. The method of any one of claims 22, 24, or 26 to 46, wherein the pomegranate extract further comprises ellagitannins (e.g., punicalagins and/or punicalins), free ellagic acid and/or anthocyanins.

48. The method of any one of claims 22, 24, or 26 to 47, wherein the pomegranate extract is a powder and further comprises ash, sugars, organic acids, and/or nitrogen, and optionally a moisture content of less than 10%.

49. The method of any one of claims 22, 24, or 26 to 48, wherein the bacterium capable of an adaptive response in the presence of pomegranate extract is a probiotic bacterium.

50. The method of any one of claims 22, 24, or 26 to 49, wherein the bacterium capable of an adaptive response in the presence of pomegranate extract is a human intestinal bacterial species.

51. The method of any one of claims 22, 24, or 26 to 50, wherein the bacterium capable of an adaptive response in the presence of pomegranate extract is from the genus Lactobacillus or Bifidobacterium.

52. The method of any one of claims 22, 24, or 26 to 51, wherein the pre-cultured bacterial population comprises cells having an increased expression of transporter genes and/or glucosidase genes, optionally dtpT, emrB and/or hsrA.

53. A method of delivering a probiotic composition to a subject, comprising administering to the subject the probiotic composition of any one of claims 1 to 18, the probiotic composition of the sachet of claim 19, or the nutritional supplement of claim 20 or 21.

54. A method of treating and/or reducing the risk of developing a disease in a subject in need thereof, comprising administering to the subject a therapeutically effective amount of the probiotic composition of any one of claims 1 to 18, the probiotic composition of the sachet of claim 19, or the nutritional supplement of claim 20 or 21, thereby treating and/or reducing the risk of developing the disease in the subject.

55. The method of claim 54, wherein the disease is a digestive disease, an inflammatory disease, cancer, or any combination thereof.


SEQUENCE LISTING (7 sequences)

SEQ ID NO: 1 - DNA, 1494 bp, Lactobacillus acidophilus
ttgggtaaacgagatacaaatacagcatttttcggacaacctaagggtttgtctacattattctttactgaaatgtgggagcgtttcagttactacggcatgcgagcaattcttttgttctacatgtactatgcagttacaaagggcggtttaggaatggatcaaactaccgctgcatcggtgatgtcgatctacggctcgcttgtttatttgtcagctttagtaggtggctggctttccgatagagtatggggatcaagaaagaccgtcttttatggtggtgtattgatcatgctaggtcacatcgtgctggctttaccagcgggtgtaggtgcattatatagttctattgctttaattgtggttggtacaggtttattaaaacctaatgtatcagatatggtagggggactttactccgtagaagatcgtcgtcgtgatgccgggtttagtatttttgtttttggtattaacctgggttcttttatcgcaccatggcttgttccttgggcagctgaaggattcggcttccatgcttttggaaatgaattgaactttcatgctggattttcgctagctgcaattggaatgttctttggattagttcaatatgttttaggtggtaaaaaatatttgcccacaagtagtctagaacctaatgatccaatcgatagaggtgatttgcttaatattattaagtgggttgttattatcgtagttgcagttgctgcaattttagctgcattagctggatttggtcaattgaacgttgataatataattactatgttgactgttttagcaattgcattgccaatttattacttcgtaatgatgctacgcagttcaaaagttactaaagttgaacgttcacgtgttaaagcttatattccattattctttgcggcagctattttctggggaatagaagaatcaggatctgttgttttagctttgtttgcagagcaaagaactgtacttcatgttggtaattggcattttacagcagccaacttccaatcacttaatcctttgtttattatgattttgacgccagtttttgtatggctatgggaacactggaagaaacaaccaagtgcgccgggtaaatttgctgctggacttgtttttgccggtttatcttatatgtggatggctttgccagggatgctttatggtacaagcggtcgagttagtcctttctggctagttggttcatggttcattgttgaaattgccgaaatgttgatttcacctattggtttatcaattacgactaagcttgcgccaaatgcatatcgttcacaaatgatgagtatgtggtttatggctgatgcgacgggtcaagcaatcaatgcacaaattgttaagttctattccactggtacagaagtacaatactttactgtcattggattagttagtgtagtcttcggtattattatgttcttattagttaagaagattaatgtattgatggaaggcgtacattaa

SEQ ID NO: 2 - DNA, 1473 bp, Lactobacillus gasseri
atggatcatcaaccaaccgatattcacggaaaagcctataatcgaaacttgcttgttctagttttaattattggatctttctgtactgtgttaaatggaaccttgcttgcaacagctcttccctcaattatgcgttcctttcatatcagtacagcaactgctgaatggctttcaactgcatttttattagttaatggagtcatgattccagtttcagcttggctaattaatcgttttggctcaagaaaaatgtatttgacagcaatgtcagttttctttattggaacagtgactgctgcagttgcgccaaattttgggactttattaactggaagaattattcaaggactcggcgtgggtgttactatgccgcttcttcaaaccatcatgctttcaatctttcccgctaataaacgcggtgctgcgatgggaacagttggtattgttattggtctcgcaccagctattggtccaaccttatctgggtggattgttgataatctttcctggcgttacttattcagtattattgctccaattgctggaattgttattattttagcgtttttcctaattaaagacgttcttccaactaagaaagaaaaaattgacgtttggtcagtgacaacttcaactattggttttggtagtttactttacggcttttcagaagctggtaataaaggatggactaaccctgaaattctaggatttattggcgtaggaattatatttgttattctttttggcatgcggcaattaaagatggcagatccatttttagatattaccgtcttcaaacatttcgaattttctctagcagctgctttaagtggtattaccaatttagcaatggttggtattgaaatggttttgccattatatattcaaaatttacgtggtgtatcagcatttcattcaggattaatgctattacctggtgccttgatgatcggaattatgtcaccaattacgggacgtttattcgataaatatggtgcacgaaaaatggcaattactggaatgaccctcttaacacttggcactattccatttgtattcttaactgcagaaagctcatatactatgattattgttttgtacgcaattagaatggtcggtgttgctttagttatgatgaacgttaccacttcaggaatgaactcattgcctttaaacaaaatctcccacggaacagcagtgaacaatacttttcgacaagttttaagttcaattggtactgctattctcgtttcaattttaactactacaaccaacaaccatatgccagcaaaagaactgcttcatacccttccacttcaatataagaatcaagcaatcaatgcaactttagatggattccgtgcttcatttgcaatgagtattctttttgccttagttgctttagttctttctttcttcttaaagaaaggcaaccgtgcacgtgaaaatcaagaagaggtgaacggataa

SEQ ID NO: 3 - DNA, 1383 bp, Lactobacillus gasseri
atgaaagtacaaactaaaaatacacttgcagttttggcaacagcattcatgtcttttgttggaattttaactgaaactagtttaaatgtaacttttcctgctatgatgaagcaattccaagtttcattggacactattcaatggattacaactggctatttgctgatgattgcgattattatgattagctcttcttatcaaaatgaacgatttacagcaagacaattgttcatttcatcagcagttgcctttataactggaagcatgatttctgcttttgcatcaagtttctacattttgcttttaggaagattaatttcagctctgggagtaggtctatgcactccgatgatgtttaatttaatcgtggaggtaatgcctaggcaaagctggggattttacatgggaattgctggtttagtaattgcaatggcaccgactttaggtcctacatttggaggcagtgtttcgtattatcttaattggcgctggattttcattattgctgcaatttttgccctctttgtctttatttccggtatttttgtaattggaagttatcatccggttcaaaagaagtcatttaactggctagcttatatatttttaagtcttagtttagtaagcttagttattggtgtaaatcaattaagtaaaggattacaaaactggcgtctttggggactattaatcttaacggttgttttgttcattttatttgttacaatttcaaaaagatctactcgaaagttgcttgacttgaaagtttttgaagataaagctttcgtttttggggcatttgcgtactttttgcttcaatttatcaatattggtgtaagttttgtattacccaattatattcaaattgttaacaaacaaagctctttaatcggtggtctggttttacttccaggaagtattattgctggacttttaaatccgtattttggccgtttatatgatcgcttaggtgcaagagtgccattatatggtggagcttttttaatggcattagggagcttcttattggctaattggggactaaacttaaatacctggatgattatctgcttttatggaattttgatgttaggacatcgaatgtcgttcagtaatacaatggcccaaagcttgaaaatagtagataataaccttaagtcagacgcaactgctgtttgtcagacagcgcaacaactagctggttcaatgggaacagcaattttagcagcaattatagctatttttcagaataagcatacagcaaaatacactactttgactgctcagggaagtatggcagcattttatttcacttttggccttggaattttgattttaatatgtgattggattatgtttaagctgagtaaagaaaataagtaa

SEQ ID NO: 4 - DNA, 555 bp, Lactobacillus acidophilus
ttgattttcaaacgtactttggttaaatacacagcagctttatcaattttctttacaggtctttcagcagttagcgttccagcaactgttcacgctgacgatgttgaaaataccactactgttgacaccaacaactcagcttcatctattgaaaccagtacacctgaatcaaattcagtaaagaaggtttcagctacaacccaaaagcgtaacgctattgttaagcttgctaagaagcaagttggtaagccttacgtttggggtgctactggcccttacggattcgactgctcaggcttaactacttatgtttacaagaatgcagctaacaagactttaccaagaactacttacggtcaaattactatcggtaaatcagtatcagtttcaactaaatctcttaagaagggtgacttattgttctggggtaactcacacgtaggtatctacattggtaacggtaagtttgttcacgctccagctccaggtcaaaatgttaagactcaaactttagcatcattctacccatcagctgctaagcgtgttatcggttaa

SEQ ID NO: 5 - DNA, 1476 bp, Lactobacillus gasseri
atgtcatttccaaagaactttttatggggcggagctactgccgcaaatcaaattgaaggtgcgtatgatgaagatggtaagggattatcagtcactgatattacaactgctggtagtttagatgcgccaagaatgttgacttacaagttgaatggcaaattagaaaaaacgccagccattcccggtgcgggcttgccagaaggtgcagttggtgcaattgatccaaatgaatattatccaaaccacgtggcaattgatttttaccaccactataaagaagacattaagatgtttgctgaaatgggctttaagaccttccgcctttcaattgcctggactagaattttccctaaaggcgatgaagaaaagccaaatcaagctggattggatttttatcgccatgtatttgaggaatgtaaaaagtacgggattgaacctttagttactatttctcactacgaagatccactatacctcagtgaaaaataccatgactggggcgatcgaaaaatgatcgatatgtatgttaaatacgcaactactttgtttgaagaatacagaggactagttaagtactggcttacatttaatgaaattaattcaacgcttttaatgcttagttcatttggtaataatgtaactgatgataaggtatatcaacatgcctaccaaaaactgcactaccaatttgtcgctagtgctcgcgcagttaagattgctcatgcattagatcctaactacgtagttggtaatatgatttgtggtattgtggactacccattaacaccagatcctaaagatattttggctaaccgtcatatgtgggaacaaaatatcttctactgcggcgatgtgcaagcaaaaggtaagtacccaacttatgctaagcgtttatggaatgaacataatgttcatttagatattactgagcaagatcgaaaagatttgcttgaagggaaagttgatatttataccttctcttactatatgtctaacattattactactcatgaagtaaaggataaagttggcggcaattttgctgctggtgttaagaacccatacttgaagtattctgaatggggctgggcaactgatccagatggtcttcaatattatcttgaagtaatgtatgatcgctacgatattccaatgatggttgttgaaaatggtcttggtgcagttgataagatcagtgatgatggtaagattcatgatgattaccgcattgattacttgagaatgcacattaaggcaatggagcgtgcaattgatgatggtgtagatttaattggttacactacttggggctgtattgacttagtttctgcaggtactggtcaaatgagtaaacgttacggctttatctatgttgacagagacgatgaaggtaagggtactttgaagagaatgccgaaagattcattctattggtacaagaaagttattgaatctgatggtaaagatttagactaa

SEQ ID NO: 6 - DNA, 1329 bp, Lactobacillus crispatus
atgaagaaaaatttaagaattgttagcgctgctgctgctgctttattagctgttgctcctgtcgctgcttcagctgtttctgttaacgctgcaagctcaagtgctgttcaaactgctaccaacattggtactgttttaccattaactgatggttctactgttaacgttaagccaaacatttcattgaacacttcagcatacgaaggtgttaaggcaaacatttcagtatcattctcagcaactgttgacggtactactgctacctctaacttcactccaaatgcttcaactattgaactttggaagaatgaaaaggataaggttacccaagtaactgatttacaacgagtaacttcatcaaacgctggtgctacttaccaagttaagatgactcaagttggcttgaacttcggttcacaaaacgctaacaagaaggttactttgactttccctgagggtgacatgttcaagaccgctgatacttctttagcacaatcacacgaagtaaaattagaccaaaacggtactattactttgccagaagtagttatgaacgtaactgctaaggacttcgctaacccagcagtagttaactggtacaacactgctactaacgcagttgtaagtactggtaacattgaacttttcgcaggttcagatgctggtaagatgaacgttgctcaagttgtttcagcaactgaaaagaagtaccacgcaagcaactacggtactaaagctaaccaagaatcaagcactatttcatacaccaacaaccttaaggatgctttaaaggctatgaacgttgatgttgatgctcaaggctggttcgttgctcctaagtcattcactttcaacatgactgctaaagctaacaacaatgatgcttcaagtaccttagctgtaactgtttcagttccaaacggtaaggacatgactgtaccaagccaaagcaagactgttatgcacaacgcattcttctatgacaagaacggcaagcgtgttggttctgacaaggtaactcgttacaactcagcaactgttgctatgaatactactactatcaacggcaaggcttactacgaagtaatcgaaaacggtaaggctactggtaagttcatcaacgctgccaacattgatggtactaagcgtactttgaagcataacgcatacgtttacaagtcttcaaagaagcgtgctaacaaggttgttcttaagaagggtactgaagtagttacctacggtggtgcttacaccttcaagaacggcaagcaatactacaagatcggtaacaatactgacaagacttacgtaaaggtttcaaacttttaa

SEQ ID NO: 7 - DNA, 597 bp, Lactobacillus crispatus
atgcaatatgtcaaaaatcatggcattcaaatggacattacagatgaagataaagaaattatgaagaaagctaaagctgatttcctagcattttcttactattcatcctgggttttatctagcgataaaattcctgatggtgaagctcctaaccattaccttaacaagggcggagttgagtccaaatatgttaagacaaacgattggggctgggcaattgatccactaggatttaggaatgcaattactacaatgtacaactactaccgtattcctattttccctattgaaaatggtatcggcctcaaggaaacttgggacggtgagcatatgattgaagatgatgagcgtattgcttatcaccgcgaccatatcaaagcaatgaaagatgctatttttgatgaaggcgccaaggtgcttggttatcttggctggggattaattgatattccaagttctcatgctgatatggaaaaacgctacggtgctgtttacgtcaacagatcaaaccacgatttaaaagatttaaaacgtgtgcctaagaaatcattctattggtttcaaaaggtacttaaagataatggagatgaattataa